(19) United States
(12) Patent Application Publication
(10) Pub. No.: US 2010/0068186 A1
(43) Pub. Date: Mar. 18, 2010

(54) High affinity telomerase t cell receptors

(75) Inventors: Bent Karsten Jakobsen, Oxfordshire (GB); Li Yi, Oxfordshire (GB)

Correspondence Address: BANNER & WITCOFF, LTD., 1100 13th STREET, N.W., SUITE 1200, WASHINGTON, DC, 20005-4051, US

(73) Assignee: AVIDEX LIMITED, Abingdon (GB)

(21) Appl. No.: 11/597,244

(22) PCT Filed: Mar. 15, 2005
(86) PCT No.: PCT/GB2005/000970

§ 371 Date: Apr. 13, 2009

(30) Foreign Application Priority Data
May 26, 2004 (GB) 0411772.7
Sep. 3, 2004 (GB) 0419644.0

(51) Int. Cl. A61K 35/12; C07K 14/705; C07K 14/52; C07K 14/55; C12N 5/00; A61K 38/17; G01N 33/566; A61P 35/04

(52) U.S. Cl. 424/93.7; 530/350; 530/351; 435/325; 514/12; 436/501

(57) ABSTRACT

The present invention provides T cell receptors (TCRs) having the property of binding to ILAKFLHWL-HLA-A*0201 and comprising at least one TCR α chain variable domain and/or at least one TCR β chain variable domain CHARACTERISED IN THAT said TCR has a K_{D} for the said ILAKFLHWL-HLA-A*0201 complex of less than or equal to 1 μM and/or has an off-rate (k_{off}) for the ILAKFLHWL-HLA-A*0201 complex of 1×10⁻³ S⁻¹ or slower.

The present invention relates to T cell receptors (TCRs) having the property of binding to ILAKFLHWL-HLA-A*0201 and comprising at least one TCR α chain variable domain and/or at least one TCR β chain variable domain CHARACTERISED IN THAT said TCR has a K_{D} for the said ILAKFLHWL-HLA-A*0201 complex of less than or equal to 1 μM and/or has an off-rate (k_{off}) for the ILAKFLHWL-HLA-A*0201 complex of 1×10⁻³ S⁻¹ or slower

### BACKGROUND TO THE INVENTION

The ILAKFLHWL peptide is derived from the catalytic sub-unit of the Telomerase protein. (See Meyerson et al., (1997) Cell 90: 785-795 and Nakamura et al., (1997) Science 277: 955-9) These studies describe the near-simultaneous discovery of the DNA and deduced amino acid sequence of the Telomerase catalytic subunit from database sequences. Both studies note that Telomerase catalytic sub-unit activity is associated with human cancer. The Class I HLA molecules of these cancerous cells, present peptides from this protein, including ILAKFLHWL. Therefore, the ILAKFLHWL-HLA-A2 complex provides a cancer marker that the TCRs of the invention can target, for example for the purpose of delivering cytotoxic agents to the cancer cells. However, for that purpose it would be desirable if the TCR had a higher affinity and/or a slower off-rate for the peptide-HLA complex than native TCRs specific for that complex.

### BRIEF DESCRIPTION OF THE INVENTION

This invention makes available for the first time TCRs having high affinity (K_{D}) of the interaction less than or equal to 1 μM, and/or a slower off-rate (k_{off}) of 1×10⁻³ S⁻¹ or slower, for the ILAKFLHWL-HLA-A*0201 complex. Such TCRs are useful, either alone or associated with a therapeutic agent, for targeting cancer cells presenting that complex

### DETAILED DESCRIPTION OF THE INVENTION

The present invention provides T-cell receptors (TCRs) having the property of binding to ILAKFLHWL-HLA-A*0201 and comprising at least one TCR α chain variable domain and/or at least one TCR β chain variable domain CHARACTERISED IN THAT said TCR has a K_{D} for the said ILAKFLHWL-HLA-A*0201 complex of less than or equal to 1 μM and/or has an off-rate (k_{off}) for the ILAKFLHWL-HLA-A*0201 complex of 1×10⁻³ S⁻¹ or slower. The K_{D} and/or (k_{off}) measurement can be made by any appropriate method. In a preferred embodiment these measurements are made using the Surface Plasmon Resonance (Biacore) method of Example 5.

For comparison a native interaction of a disulfide-linked soluble variant of the native ILA TCR (see SEQ ID NO: 9 for TCR α chain and SEQ ID NO: 10 for TCR β chain) and the ILAKFLHWL-HLA-A*0201 complex has a K_{D} of approximately 25 μM, an off-rate (k_{off}) of 1.1×10⁻¹ S⁻¹ and a half-life of 0.18 minutes as measured by the Biacore-base method of Example 5.

The native ILA TCR has the following Valpha chain and Vbeta chain gene usage:

Alpha chain—TRAV22
Beta chain:—TRBV 6.5

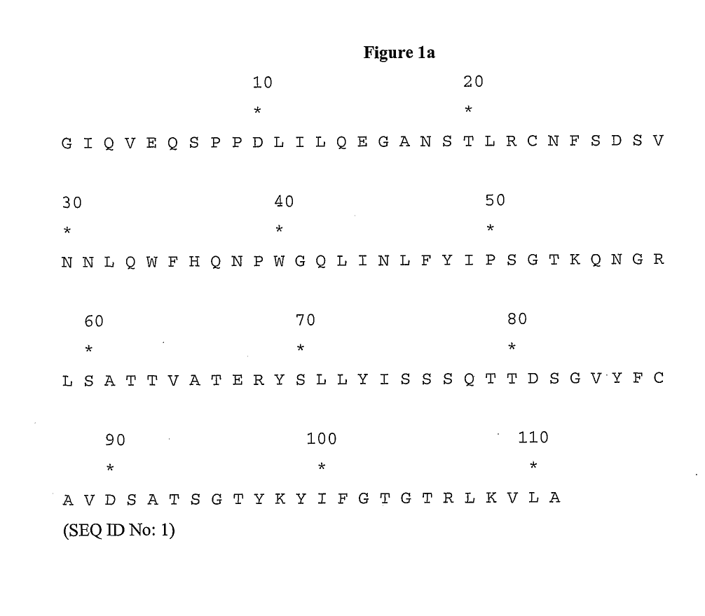

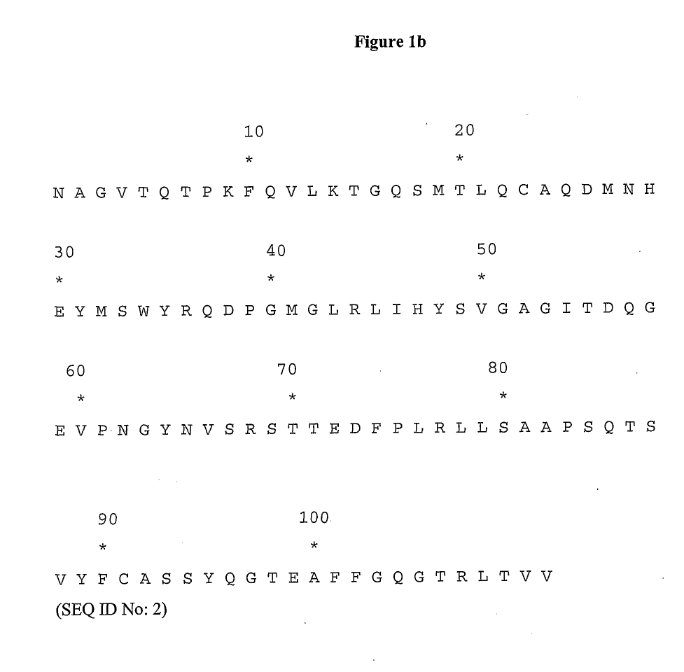

The ILA TCR can be used as a template into which the various mutations that cause high affinity and/or a slow off-rate for the interaction between TCRs of the invention and the ILAKFLHWL-HLA-A*0201 complex can be introduced. Further embodiments of the inventions include TCRs which are mutated relative to the native ILA TCR α chain variable domain (see FIG. 1a and SEQ ID No: 1) and/or β chain variable domain (see FIG. 1b and SEQ ID NO: 2) in at least one complementarity determining region (CDR) and/or variable domain framework region thereof. It is also contemplated that other hypervariable regions in the variable domains of the TCRs of the invention, such as the hypervariable 4 (HV4) regions, may be mutated so as to produce a high affinity mutant.

Native TCRs exist in heterodimeric αβ or γδ forms. However, recombinant TCRs consisting of a single TCR α or TCR β chain have previously been shown to bind to peptide MHC molecules. In the case where the product of the present invention comprises only a TCR α chain or a TCR β chain, such products are treated as TCRs for the purposes of the invention notwithstanding that the more normal usage of the term TCR relates to heterodimers of α and β chains or single chain constructs comprising α and β portions, especially α and β variable regions. In addition, the term TCR as used herein is to be understood as including αα and ββ homodimers.

In one embodiment the TCR of the invention comprise both an α chain variable domain and an TCR β chain variable domain.

As will be obvious to those skilled in the art the mutation(s) in the TCR α chain sequence and/or TCR β chain sequence may be one or more of substitution(s), deletion(s) or insertion(s). These mutations can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) mutagenesis and restriction enzyme-based cloning see (Sambrook & Russell, (2001) Molecular Cloning—A Laboratory Manual (3^{rd} Ed.) CSHL Press) Further information on LIC procedures can be found in (Rashtchian, (1995) Curr Opin Biotechnol 6 (1): 30-6)

It should be noted that any αβ TCR that comprises similar Valpha and Vbeta gene usage and therefore amino acid sequence to that of the ILA TCR could make a convenient template TCR. It would then be possible to introduce into the DNA encoding one or both of the variable domains of the template αβ TCR the changes required to produce the mutated high affinity TCRs of the invention. As will be obvious to those skilled in the art, the necessary mutations could be introduced by a number of methods, for example site-directed mutagenesis.

The TCRs of the invention include those in which one or more of the TCR alpha chain variable domain amino acids corresponding to those listed below are mutated relative to the amino acid occurring at these positions in the sequence provided for the native ILA TCR alpha chain variable domain in FIG. 1a and SEQ ID No: 1.

Unless stated to the contrary, the TCR amino acid sequences herein are generally provided without an N-terminal methionine (Met or M) residue. As will be known to those skilled in the art this residue is often added during the production of recombinant proteins. As will also be known to those skilled in the art it may be possible to truncate the sequences provided at the C-terminus and/or N-terminus thereof, by 1, 2, 3, 4, 5 or more residues, without substantially affecting the pMHC binding characteristics of the TCR, all such trivial variants are encompassed by the present invention.

As used herein the term “variable domain” is understood to encompass all amino acids of a given TCR which are not included within the constant domain as encoded by the TRAC gene for TCR α chains and either the TRBC1 or TRBC2 for TCR β chains. (T cell receptor Factsbook, (2001) LeFranc and LeFranc, Academic Press, ISBN 0-12-441352-8)

Embodiments of the invention include mutated TCRs which comprise mutation of one or more of alpha chain variable domain amino acids corresponding to 94S, 95G, 96T, 97Y and 98K for example the amino acids:
94A/Q/T/G/R/M/H/K//E/N or L
95L/P/I/V or M
96P or M
97F
98G or A

The numbering used is the same as that shown in FIG. 1a and SEQ ID No: 1.

Embodiments of the invention also include TCRs which comprise mutation of one or more of the TCR beta chain variable domain amino acids corresponding to those listed below, are relative to the amino acid occurring at these positions in the sequence provided for the native ILA TCR alpha chain variable domain of the native ILA TCR beta chain in FIG. 1b and SEQ ID No: 2. The amino acids referred to which may be mutated are 18M, 49V, 50G, 51A, 52G, 53I, 92A, 93S, 94S and 95Y, for example:
19V
50I/L or R
51H/C or W
52P/W/E or V
53E/S or F
54Y/V/C/E or D
92G
93I/L/ or V
94Q
95P

The numbering used is the same as that shown in FIG. 1b and SEQ ID No: 2.

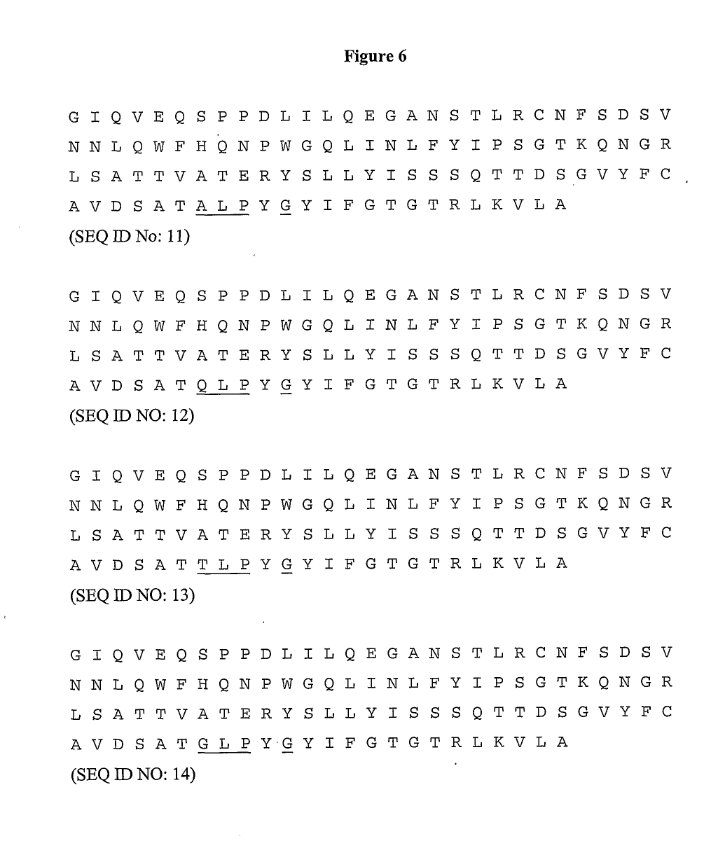

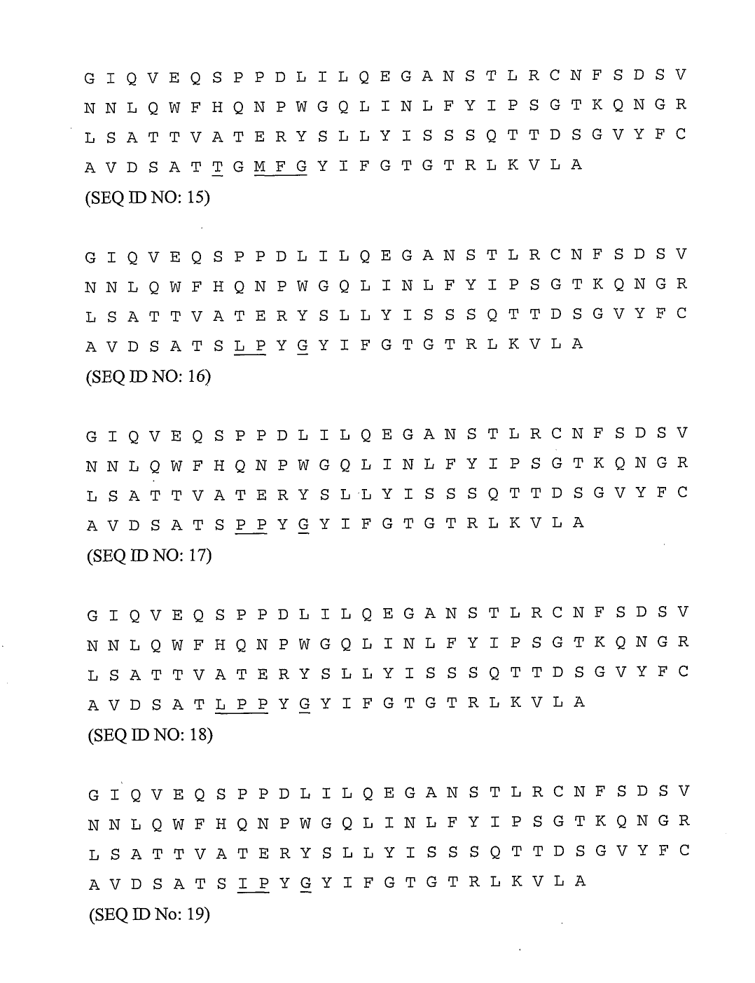

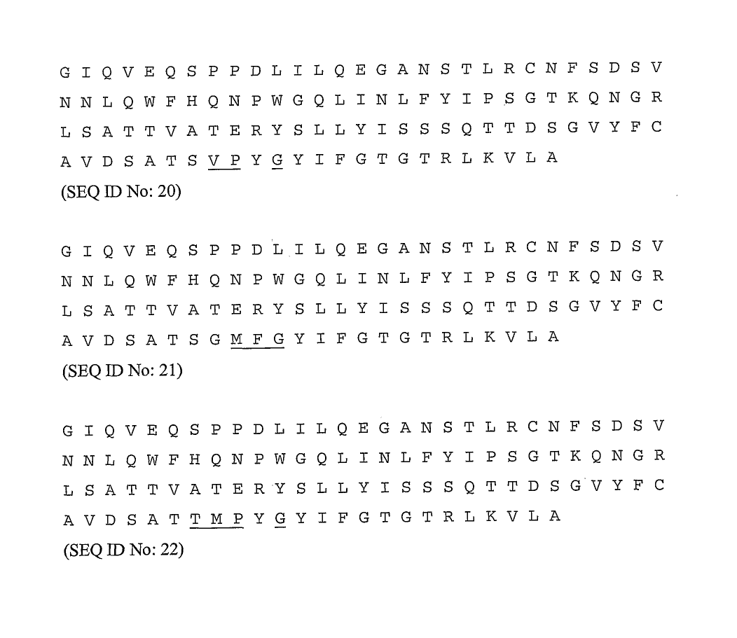

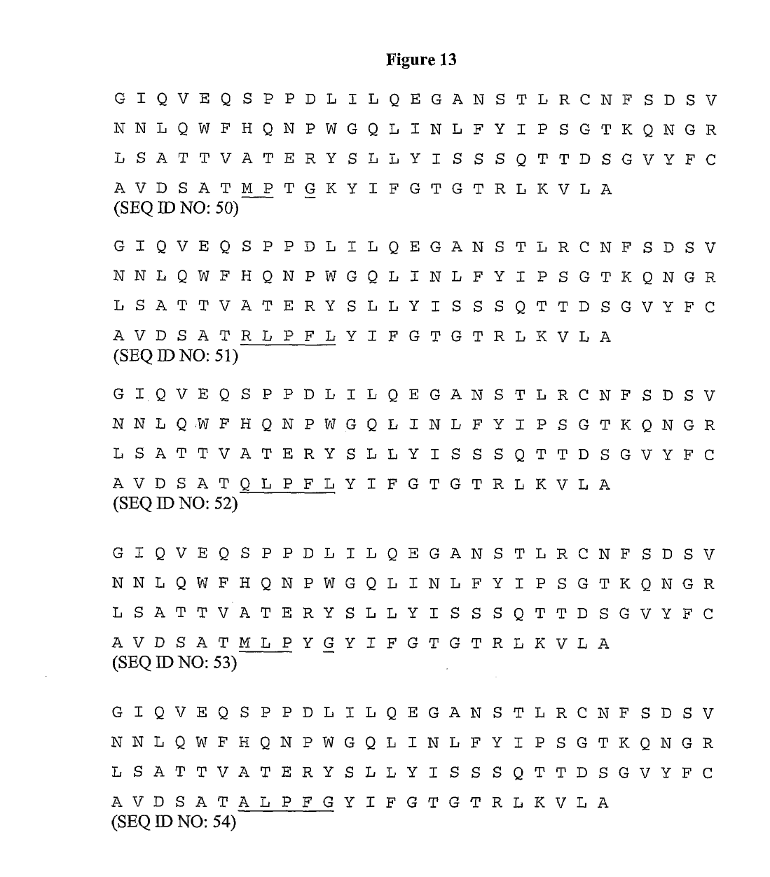

Preferred embodiments of the invention are provided by TCRs comprising one of the mutated alpha chain variable domain amino acid sequences shown in FIG. 6 or 13 (SEQ ID Nos: 11 to 22 or 50 to 60). Phenotypically silent variants of such TCRs also form part of this invention.

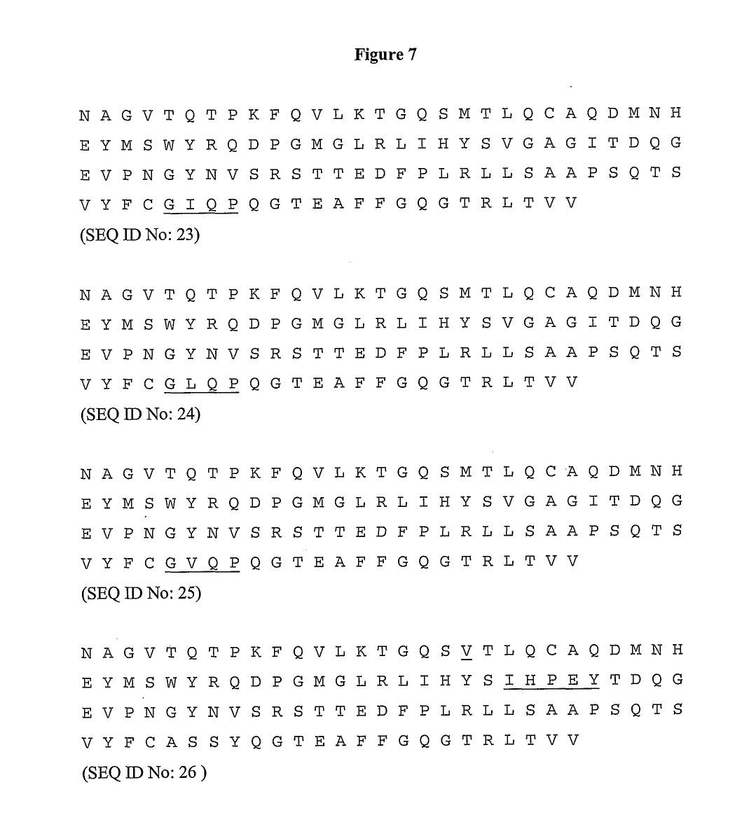

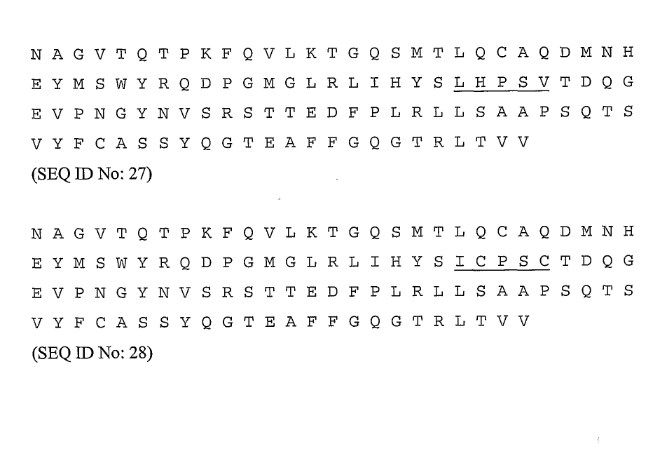

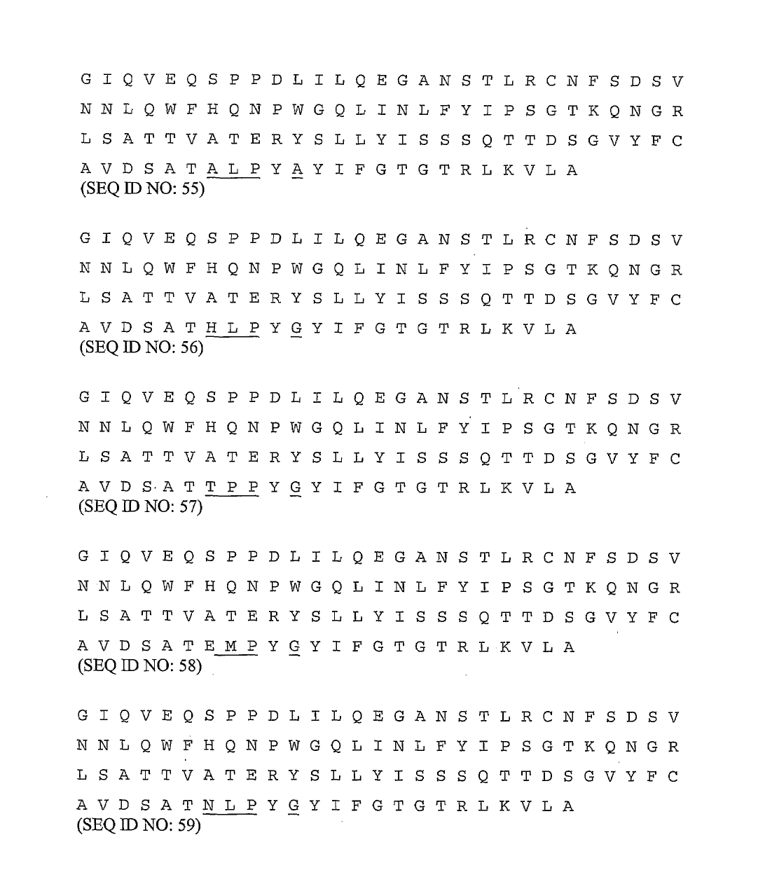

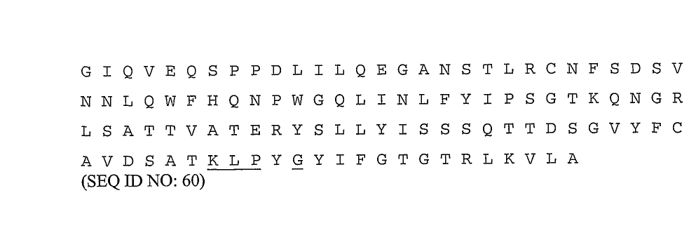

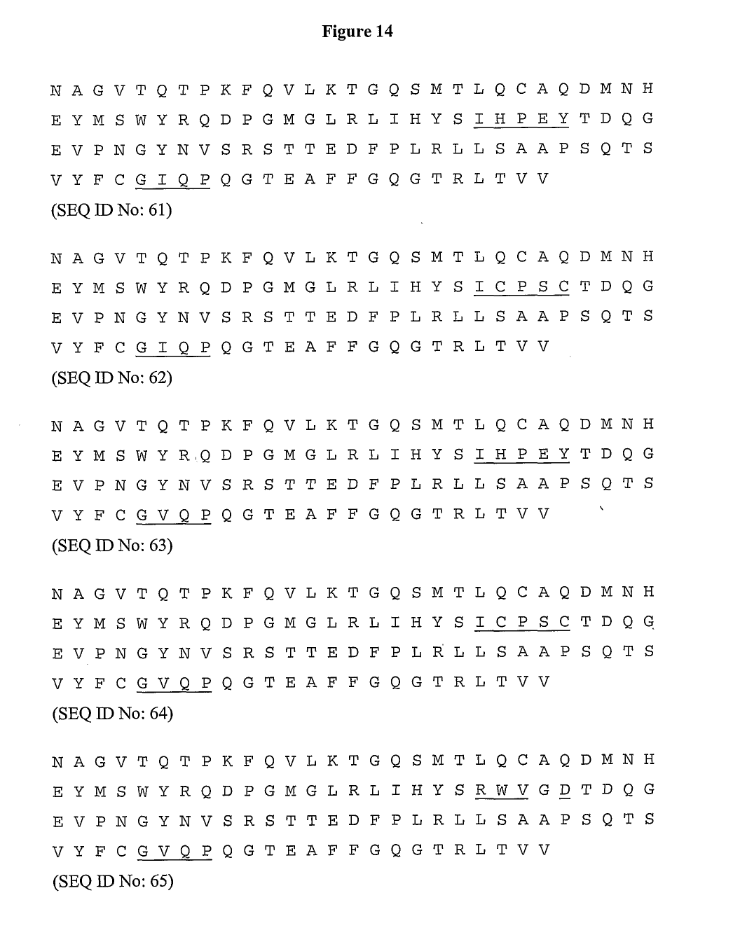

Additional preferred embodiments of the invention are provided by TCRs comprising one of the mutated beta chain variable domain amino acid sequences shown in FIG. 7 or 14. (SEQ ID Nos: 23 to 31 or 61 to 70). Phenotypically silent variants of such TCRs also form part of this invention.

Native TCRs exist in heterodimeric αβ or γδ forms. However, recombinant TCRs consisting of αα or ββ homodimers have previously been shown to bind to peptide MHC molecules. Therefore, one embodiment of the invention is provided by TCR αα or TCR ββ homodimers.

Further preferred embodiments are provided by TCRs of the invention comprising the alpha chain variable domain amino acid sequence and the beta chain variable domain amino acid sequence combinations listed below, phenotypically silent variants of such TCRs also form part of this invention:

| Alpha chain variable | Beta chain variable |
|---|---|
| domain sequence, | domain sequence, |
| SEQ ID NO: | SEQ ID NO: |
| 1 | 23 |
| 1 | 24 |
| 1 | 25 |
| 1 | 26 |
| 1 | 27 |
| 1 | 28 |
| 1 | 29 |
| 1 | 30 |
| 1 | 31 |
| 11 | 2 |
| 12 | 2 |
| 13 | 2 |
| 14 | 2 |
| 15 | 2 |
| 16 | 2 |
| 17 | 2 |
| 18 | 2 |
| 19 | 2 |
| 20 | 2 |
| 21 | 2 |
| 22 | 2 |
| 50 | 2 |
| 51 | 2 |
| 52 | 2 |
| 53 | 2 |
| 54 | 2 |
| 55 | 2 |
| 56 | 2 |
| 57 | 2 |
| 58 | 2 |
| 59 | 2 |
| 60 | 2 |
| 17 | 25 |
| 17 | 23 |
| 12 | 23 |
| 16 | 23 |
| 14 | 23 |
| 12 | 25 |
| 15 | 23 |
| 19 | 23 |
| 20 | 23 |
| 11 | 61 |
| 11 | 62 |
| 11 | 63 |
| 11 | 64 |
| 11 | 65 |
| 14 | 65 |
| 16 | 66 |
| 22 | 65 |
| 20 | 31 |
| 17 | 31 |
| 17 | 66 |
| 18 | 65 |
| 19 | 65 |
| 17 | 65 |
| 21 | 65 |
| 11 | 31 |
| 19 | 31 |
| 16 | 31 |
| 12 | 31 |
| 11 | 66 |
| 17 | 67 |
| 11 | 67 |
| 15 | 66 |
| 15 | 68 |
| 11 | 67 |
| 15 | 8 |
| 11 | 69 |
| 15 | 70 |
| 15 | 69 |
| 17 | 69 |
| 11 | 70 |
| 17 | 70 |
| 11 | 28 |
| 11 | 23 |

Preferred embodiments provide a TCR of the invention comprising:

the alpha chain variable domain shown in the SEQ ID NO: 11 and the beta chain variable domain shown in the SEQ ID NO: 23, or phenotypically silent variants thereof;
the alpha chain variable domain shown in the SEQ ID NO: 11 and the beta chain variable domain shown in the SEQ ID NO: 64, or phenotypically silent variants thereof;
the alpha chain variable domain shown in the SEQ ID NO: 11 and the beta chain variable domain shown in the SEQ ID NO: 65, phenotypically silent variants thereof;

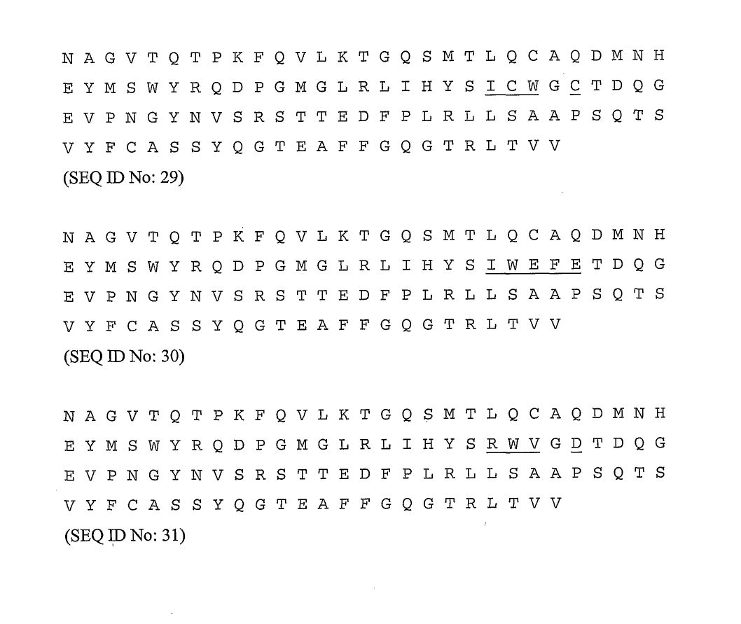

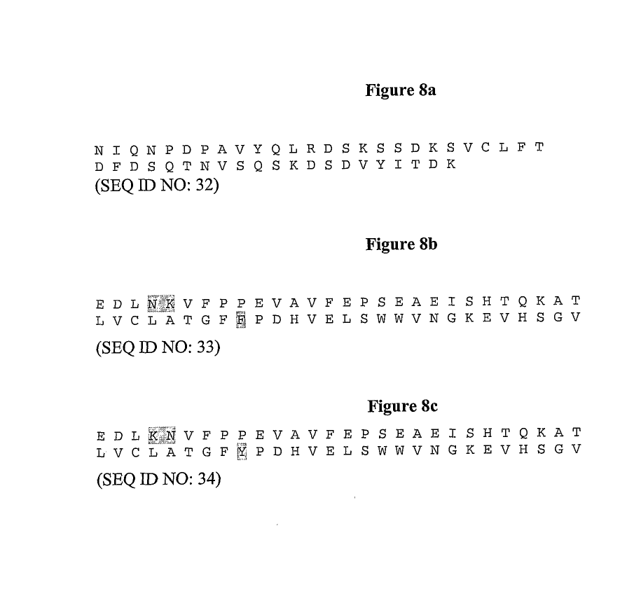

In another preferred embodiment TCRs of the invention comprising the variable domain combinations detailed above further comprise the alpha chain constant region amino acid sequence shown in FIG. 8a (SEQ ID NO: 32) and one of the beta chain amino acid constant region sequences shown in FIGS. 8b and 8c (SEQ ID NOs: 33 and 34) or phenotypically silent variants thereof.

As used herein the term “phenotypically silent variants” is understood to refer to those TCRs which have a K_{D} for the said ILAKFLHWL (SEQ ID NO: 37)—HLA-A*0201 complex of less than or equal to 1 μM and/or has an off-rate (k_{off}) of 1×10⁻³ S⁻¹ or slower. For example, as is known to those skilled in the art, it may be possible to produce TCRs that incorporate changes in the constant and/or variable domains thereof compared to those detailed above without altering the affinity and/or off-rate for the interaction with the ILAKFLHWL-HLA-A*0201 complex. Such trivial variants are included in the scope of this invention. Those TCRs in which one or more conservative substitutions have been made also form part of this invention.

A suitable scTCR form comprises a first segment constituted by an amino acid sequence corresponding to a TCR α chain variable domain, a second segment constituted by an amino acid sequence corresponding to a TCR β chain variable domain sequence fused to the N terminus of an amino acid sequence corresponding to a TCR β chain constant domain extracellular sequence, and a linker sequence linking the C terminus of the first segment to the N terminus of the second segment.

Alternatively the first segment may be constituted by an amino acid sequence corresponding to a TCR β chain variable domain, the second segment may be constituted by an amino acid sequence corresponding to a TCR α chain variable domain sequence fused to the N terminus of an amino acid sequence corresponding to a TCR α chain constant domain extracellular sequence

The above scTCRs may further comprise a disulfide bond between the first and second chains, said disulfide bond being one which has no equivalent in native αβT cell receptors, and wherein the length of the linker sequence and the position of the disulfide bond being such that the variable domain sequences of the first and second segments are mutually orientated substantially as in native αβ T cell receptors.

More specifically the first segment may be constituted by an amino acid sequence corresponding to a TCR α chain variable domain sequence fused to the N terminus of an amino acid sequence corresponding to a TCR α chain constant domain extracellular sequence, the second segment may be constituted by an amino acid sequence corresponding to a TCR β chain variable domain fused to the N terminus of an amino acid sequence corresponding to TCR β chain constant domain extracellular sequence, and a disulfide bond may be provided between the first and second chains, said disulfide bond being one which has no equivalent in native αβ T cell receptors.

In the above scTCR forms, the linker sequence may link the C terminus of the first segment to the N terminus of the second segment, and may have the formula —PGGG—(SGGGG)ₙ-P— wherein n is 5 or 6 and P is proline, G is glycine and S is serine:

(SEQ ID NO: 35)

PGGG-SGGGGSGGGGSGGGGSGGGGSGGGG-P

(SEQ ID NO: 36)

PGGG-SGGGGSGGGGSGGGGSGGGGSGGGGSGGGG-P

A suitable dTCR form of the TCRs of the present invention comprises a first polypeptide wherein a sequence corresponding to a TCR α chain variable domain sequence is fused to the N terminus of a sequence corresponding to a TCR α chain constant domain extracellular sequence, and a second polypeptide wherein a sequence corresponding to a TCR β chain variable domain sequence fused to the N terminus a sequence corresponding to a TCR β chain constant domain extracellular sequence, the first and second polypeptides being linked by a disulfide bond which has no equivalent in native αβ T cell receptors.

The first polypeptide may comprise a TCR α chain variable domain sequence is fused to the N terminus of a sequence corresponding to a TCR α chain constant domain extracellular sequence, and a second polypeptide wherein a sequence corresponding to a TCR β chain variable domain sequence is fused to the N terminus a sequence corresponding to a TCR β chain constant domain extracellular sequence, the first and second polypeptides being linked by a disulfide bond between cysteine residues substituted for Thr 48 of exon 1 of TRAC*01 and Ser 57 of exon 1 of TRBC1*01 or TRBC2*01 or the non-human equivalent thereof. (“TRAC” etc. nomenclature herein as per T cell receptor Factsbook, (2001) LeFranc and LeFranc, Academic Press, ISBN 0-12-441352-8)

The dTCR or scTCR form of the TCRs of the invention may have amino acid sequences corresponding to human αβ TCR extracellular constant and variable domain sequences, and a disulfide bond may link amino acid residues of the said constant domain sequences, which disulfide bond has no equivalent in native TCRs. The disulfide bond is between cysteine residues corresponding to amino acid residues whose β carbon atoms are less than 0.6 nm apart in native TCRs, for example between cysteine residues substituted for Thr 48 of exon 1 of TRAC*01 and Ser 57 of exon 1 of TRBC1*01 or TRBC2*01 or the non-human equivalent thereof. Other sites where cysteines can be introduced to form the disulfide bond are the following residues in exon 1 of TRAC*01 for the TCR α chain and TRBC1*01 or TRBC2*01 for the TCR β chain:

| | | Native β carbon |
|---|---|---|
| TCR α chain | TCR β chain | separation (nm) |
| Thr 45 | Ser 77 | 0.533 |
| Tyr 10 | Ser 17 | 0.359 |
| Thr 45 | Asp 59 | 0.560 |
| Ser 15 | Glu 15 | 0.59 |

In addition to the non-native disulfide bond referred to above, the dTCR or scTCR form of the TCRs of the invention may include a disulfide bond between residues corresponding to those linked by a disulfide bond in native TCRs.

The dTCR or scTCR form of the TCRs of the invention preferably does not contain a sequence corresponding to transmembrane or cytoplasmic sequences of native TCRs.

Preferred embodiments of the invention provide a soluble TCR consisting of:

the alpha chain amino acid sequence of SEQ ID NO: 71 and beta chain amino acid sequence SEQ ID NO: 73:
the alpha chain amino acid sequence of SEQ ID NO: 71 and beta chain amino acid sequence SEQ ID NO: 76;
the alpha chain amino acid sequence of SEQ ID NO: 71 and beta chain amino acid sequence SEQ ID NO: 74;
the alpha chain amino acid sequence of SEQ ID NO: 71 and beta chain amino acid sequence SEQ ID NO: 77;
the alpha chain amino acid sequence of SEQ ID NO: 71 and beta chain amino acid sequence SEQ ID NO: 75;
the alpha chain amino acid sequence of SEQ ID NO: 71 and beta chain amino acid sequence SEQ ID NO: 78;
SEQ ID NOs: 71, and 73-78 have been provided in a form which includes the N-terminal methionine (M).

### PEGylated TCR Monomers

In one particular embodiment a TCR of the invention is associated with at least one polyalkylene glycol chain(s). This association may be cause in a number of ways known to those skilled in the art. In a preferred embodiment the polyalkylene chain(s) is/are covalently linked to the TCR. In a further embodiment the polyethylene glycol chains of the present aspect of the invention comprise at least two polyethylene repeating units.

### Multivalent TCR Complexes

One aspect of the invention provides a multivalent TCR complex comprising at least two TCRs of the invention. In one embodiment of this aspect, at least two TCR molecules are linked via linker moieties to form multivalent complexes. Preferably the complexes are water soluble, so the linker moiety should be selected accordingly. Furthermore, it is preferable that the linker moiety should be capable of attachment to defined positions on the TCR molecules, so that the structural diversity of the complexes formed is minimised. One embodiment of the present aspect is provided by a TCR complex of the invention wherein the polymer chain or peptidic linker sequence extends between amino acid residues of each TCR which are not located in a variable region sequence of the TCR.

Since the complexes of the invention may be for use in medicine, the linker moieties should be chosen with due regard to their pharmaceutical suitability, for example their immunogenicity.

Examples of linker moieties which fulfil the above desirable criteria are known in the art, for example the art of linking antibody fragments.

There are two classes of linker that are preferred for use in the production of multivalent TCR molecules of the present invention. A TCR complex of the invention in which the TCRs are linked by a polyalkylene glycol chain provides one embodiment of the present aspect.

The first are hydrophilic polymers such as polyalkylene glycols. The most commonly used of this class are based on polyethylene glycol or PEG, the structure of which is shown below.

HOCH₂CH₂O(CH₂CH₂O)ₙ—CH₂CH₂OH

Wherein n is greater than two. However, others are based on other suitable, optionally substituted, polyalkylene glycols include polypropylene glycol, and copolymers of ethylene glycol and propylene glycol.

Such polymers may be used to treat or conjugate therapeutic agents, particularly polypeptide or protein therapeutics, to achieve beneficial changes to the PK profile of the therapeutic, for example reduced renal clearance, improved plasma half-life, reduced immunogenicity, and improved solubility. Such improvements in the PK profile of the PEG-therapeutic conjugate are believe to result from the PEG molecule or molecules forming a ‘shell’ around the therapeutic which sterically hinders the reaction with the immune system and reduces proteolytic degradation. (Casey et al, (2000) Tumor Targetting 4 235-244) The size of the hydrophilic polymer used my in particular be selected on the basis of the intended therapeutic use of the TCR complex. Thus for example, where the product is intended to leave the circulation and penetrate tissue, for example for use in the treatment of a tumour, it may be advantageous to use low molecular weight polymers in the order of 5 KDa. There are numerous review papers and books that detail the use of PEG and similar molecules in pharmaceutical formulations. For example, see Harris (1992) Polyethylene Glycol Chemistry—Biotechnical and Biomedical Applications, Plenum, New York, N.Y. or Harris & Zalipsky (1997) Chemistry and Biological Applications of Polyethylene Glycol ACS Books, Washington, D.C.

The polymer used can have a linear or branched conformation. Branched PEG molecules, or derivatives thereof, can be induced by the addition of branching moieties including glycerol and glycerol oligomers, pentaerythritol, sorbitol and lysine.

Usually, the polymer will have a chemically reactive group or groups in its structure, for example at one or both termini, and/or on branches from the backbone, to enable the polymer to link to target sites in the TCR. This chemically reactive group or groups may be attached directly to the hydrophilic polymer, or there may be a spacer group/moiety between the hydrophilic polymer and the reactive chemistry as shown below:
Reactive chemistry-Hydrophilic polymer-Reactive chemistry
Reactive chemistry-Spacer-Hydrophilic polymer-Spacer-Reactive chemistry

The spacer used in the formation of constructs of the type outlined above may be any organic moiety that is a non-reactive, chemically stable, chain, Such spacers include, by are not limited to the following:
—(CH₂)ₙ— wherein n=2 to 5
—(CH₂)₃NHCO(CH₂)₂

A TCR complex of the invention in which a divalent alkylene spacer radical is located between the polyalkylene glycol chain and its point of attachment to

a TCR of the complex provides a further embodiment of the present aspect.

A TCR complex of the invention in which the polyalkylene glycol chain comprises at least two polyethylene glycol repeating units provides a further embodiment of the present aspect.

There are a number of commercial suppliers of hydrophilic polymers linked, directly or via a spacer, to reactive chemistries that may be of use in the present invention. These suppliers include Nektar Therapeutics (CA, USA), NOF Corporation (Japan), Sunbio (South Korea) and Enzon Pharmaceuticals (NJ, USA).

Commercially available hydrophilic polymers linked, directly or via a spacer, to reactive chemistries that may be of use in the present invention include, but are not limited to, the following:

| PEG linker | | Catalogue |
|---|---|---|
| Description | Source of PEG | Number |
| TCR Monomer attachment | | |
| 5K linear (Maleimide) | Nektar | 2D2MOHO1 |
| 20K linear (Maleimide) | Nektar | 2D2MOPO1 |
| 20K linear (Maleimide) | NOF Corporation | SUNBRIGHT |
| | | ME-200MA |
| 20K branched (Maleimide) | NOF Corporation | SUNBRIGHT |
| | | GL2-200MA |
| 30K linear (Maleimide) | NOF Corporation | SUNBRIGHT |
| | | ME-300MA |
| 40K branched PEG (Maleimide) | Nektar | 2D3XOTO1 |
| 5K-NP linear (for Lys attachment) | NOF Corporation | SUNBRIGHT |
| | | MENP-50H |
| 10K-NP linear (for Lys attachment) | NOF Corporation | SUNBRIGHT |
| | | MENP-10T |
| 20K-NP linear (for Lys attachment) | NOF Corporation | SUNBRIGHT |
| | | MENP-20T |
| TCR dimer linkers | | |
| 3.4K linear (Maleimide) | Nektar | 2D2DOFO2 |
| 5K forked (Maleimide) | Nektar | 2D2DOHOF |
| 10K linear (with orthopyridyl ds- | Sunbio | |
| linkers in place of Maleimide) | | |
| 20K forked (Maleimide) | Nektar | 2D2DOPOF |
| 20K linear (Maleimide) | NOF Corporation | |
| 40K forked (Maleimide) | Nektar | 2D3XOTOF |
| Higher order TCR multimers | | |
| 15K, 3 arms, Mal₃ (for trimer) | Nektar | OJOONO3 |
| 20K, 4 arms, Mal₄ (for tetramer) | Nektar | OJOOPO4 |
| 40 K, 8 arms, Mal₈ (for octamer) | Nektar | OJOOTO8 |

A wide variety of coupling chemistries can be used to couple polymer molecules to protein and peptide therapeutics. The choice of the most appropriate coupling chemistry is largely dependant on the desired coupling site. For example, the following coupling chemistries have been used attached to one or more of the termini of PEG molecules (Source: Nektar Molecular Engineering Catalogue 2003):
N-maleimide
Vinyl sulfone
Benzotriazole carbonate
Succinimidyl proprionate
Succinimidyl butanoate
Thio-ester
Acetaldehydes
Acrylates
Biotin
Primary amines

As stated above non-PEG based polymers also provide suitable linkers for multimerising the TCRs of the present invention. For example, moieties containing maleimide termini linked by aliphatic chains such as BMH and BMOE (Pierce, products Nos. 22330 and 22323) can be used.

Peptidic linkers are the other class of TCR linkers. These linkers are comprised of chains of amino acids, and function to produce simple linkers or multimerisation domains onto which TCR molecules can be attached. The biotin/streptavidin system has previously been used to produce TCR tetramers (see WO/99/60119) for in-vitro binding studies. However, streptavidin is a microbially-derived polypeptide and as such not ideally suited to use in a therapeutic.

A TCR complex of the invention in which the TCRs are linked by a peptidic linker derived from a human multimerisation domain provides a further embodiment of the present aspect.

There are a number of human proteins that contain a multimerisation domain that could be used in the production of multivalent TCR complexes. For example the tetramerisation domain of p53 which has been utilised to produce tetramers of scFv antibody fragments which exhibited increased serum persistence and significantly reduced off-rate compared to the monomeric scFV fragment. (Willuda et al. (2001) J. Biol. Chem. 276 (17) 14385-14392) Haemoglobin also has a tetramerisation domain that could potentially be used for this kind of application.

A multivalent TCR complex of the invention comprising at least two TCRs provides a final embodiment of this aspect, wherein at least one of said TCRs is associated with a therapeutic agent.

In one aspect a TCR (or multivalent complex thereof) of the present invention may alternatively or additionally comprise a reactive cysteine at the C-terminal or N-terminal of the alpha or beta chains thereof.

### Diagnostic and Therapeutic Use

In one aspect the TCR of the invention may be associated with a therapeutic agent or detectable moiety. For example, said therapeutic agent or detectable moiety may be covalently linked to the TCR.

In one embodiment of the invention said therapeutic agent or detectable moiety is covalently linked to the C-terminus of one or both TCR chains.

In one aspect the scTCR or one or both of the dTCR chains of TCRs of the present invention may be labelled with an detectable moiety, for example a label that is suitable for diagnostic purposes. Such labelled TCRs are useful in a method for detecting a ILAKFLHWL-HLA-A*0201 complex which method comprises contacting the TCR ligand with a TCR (or a multimeric high affinity TCR complex) which is specific for the TCR ligand; and detecting binding to the TCR ligand. In tetrameric TCR complexes formed for example, using biotinylated heterodimers, fluorescent streptavidin can be used to provide a detectable label. Such a fluorescently-labelled TCR tetramer is suitable for use in FACS analysis, for example to detect antigen presenting cells carrying the ILAKFLHWL-HLA-A*0201 complex for which these high affinity TCRs are specific.

Another manner in which the soluble TCRs of the present invention may be detected is by the use of TCR-specific antibodies, in particular monoclonal antibodies. There are many commercially available anti-TCR antibodies, such as αF1 and βF1, which recognise the constant domains of the α and β chains, respectively.

In a further aspect a TCR (or multivalent complex thereof) of the present invention may alternatively or additionally be associated with (e.g. covalently or otherwise linked to) a therapeutic agent which may be, for example, a toxic moiety for use in cell killing, or an immune effector molecule such as an interleukin or a cytokine. A multivalent TCR complex of the invention may have enhanced binding capability for a TCR ligand compared to a non-multimeric wild-type or T cell receptor heterodimer of the invention. Thus, the multivalent TCR complexes according to the invention are particularly useful for tracking or targeting cells presenting particular antigens in vitro or in vivo, and are also useful as intermediates for the production of further multivalent TCR complexes having such uses. These TCRs or multivalent TCR complexes may therefore be provided in a pharmaceutically acceptable formulation for use in vivo.

The invention also provides a method for delivering a therapeutic agent to a target cell, which method comprises contacting potential target cells with a TCR or multivalent TCR complex in accordance with the invention under conditions to allow attachment of the TCR or multivalent TCR complex to the target cell, said TCR or multivalent TCR complex being specific for the ILAKFLHWL-HLA-A*0201 complex and having the therapeutic agent associated therewith.

In particular, the soluble TCR or multivalent TCR complex of the present invention can be used to deliver therapeutic agents to the location of cells presenting a particular antigen. This would be useful in many situations and, in particular, against tumours. A therapeutic agent could be delivered such that it would exercise its effect locally but not only on the cell it binds to. Thus, one particular strategy envisages anti-tumour molecules linked to TCRs or multivalent TCR complexes according to the invention specific for tumour antigens.

Many therapeutic agents could be employed for this use, for instance radioactive compounds, enzymes (perforin for example) or chemotherapeutic agents (cis-platin for example). To ensure that toxic effects are exercised in the desired location the toxin could be inside a liposome linked to streptavidin so that the compound is released slowly. This will prevent damaging effects during the transport in the body and ensure that the toxin has maximum effect after binding of the TCR to the relevant antigen presenting cells.

Other suitable therapeutic agents include:
small molecule cytotoxic agents, i.e. compounds with the ability to kill mammalian cells having a molecular weight of less than 700 daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolmide, topotecan, trimetreate glucuronate, auristatin E vincristine and doxorubicin;
peptide cytotoxins, i.e. proteins or fragments thereof with the ability to kill mammalian cells. Including but not limited to, ricin, diphtheria toxin, pseudomonas bacterial exotoxin A, DNAase and RNAase;
radio-nuclides, i.e. unstable isotopes of elements which decay with the concurrent emission of one or more of α or β particles, or γ rays. including but not limited to, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
prodrugs, including but not limited to, antibody directed enzyme pro-drugs;
immuno-stimulants, i.e. moieties which stimulate immune response. Including but not limited to, cytokines such as IL-2 and IFN, Superantigens and mutants thereof, TCR-HLA fusions and chemokines such as IL-8, platelet factor 4, melanoma growth stimulatory protein, etc, antibodies or fragments thereof, complement activators, xenogeneic protein domains, allogeneic protein domains, viral/bacterial protein domains, viral/bacterial peptides and anti-T cell determinant antibodies (e.g. anti-CD3 or anti-CD28).

Soluble TCRs or multivalent TCR complexes of the invention may be linked to an enzyme capable of converting a prodrug to a drug. This allows the prodrug to be converted to the drug only at the site where it is required (i.e. targeted by the sTCR).

It is expected that the high affinity ILAKFLHWL (SEQ ID NO: 37)—HLA-A*0201 specific TCRs disclosed herein may be used in methods for the diagnosis and treatment of cancer.

For cancer treatment, the localisation in the vicinity of tumours or metastasis would enhance the effect of toxins or immunostimulants. For vaccine delivery, the vaccine antigen could be localised in the vicinity of antigen presenting cells, thus enhancing the efficacy of the antigen. The method can also be applied for imaging purposes.

One embodiment is provided by an isolated cell presenting a TCR of the invention. For example, said cell may be a T cell.

Further embodiments of the invention are provided by a pharmaceutical composition comprising:

a TCR or a multivalent TCR complex of the invention (optionally associated with a therapeutic agent), or a plurality of cells presenting at least one TCR of the invention, together with a pharmaceutically acceptable carrier;

The invention also provides a method of treatment of cancer comprising administering to a subject suffering such cancer disease an effective amount of a TCR or a multivalent TCR complex of the invention (optionally associated with a therapeutic agent), or a plurality of cells presenting at least one TCR of the invention. In a related embodiment the invention provides for the use of a TCR or a multivalent TCR complex of the invention (optionally associated with a therapeutic agent), or a plurality of cells presenting at least one TCR of the invention, in the preparation of a composition for the treatment of cancer.

Therapeutic or imaging TCRs in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example parenteral, transdermal or via inhalation, preferably a parenteral (including subcutaneous, intramuscular, or, most preferably intravenous) route. Such compositions may be prepared by any method known in the art of pharmacy, for example by admixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

### Additional Aspects

A scTCR or dTCR (which preferably is constituted by constant and variable sequences corresponding to human sequences) of the present invention may be provided in substantially pure form, or as a purified or isolated preparation. For example, it may be provided in a form which is substantially free of other proteins.

The invention also provides a method of producing a high affinity TCR having the property of binding to ILAKFLHWL-HLA-A*0201.CHARACTERISED IN THAT the TCR (i) comprises at least one TCR α chain variable domain and/or at least one TCR β chain variable domain and (ii) has a K_{D} for the said ILAKFLHWL-HLA-A*0201 complex of less than or equal to 1 μM and/or an off-rate (k_{off}) for the ILAKFLHWL-HLA-A*0201 complex of 1×10⁻³ S⁻¹ or slower, wherein the method comprises:
(a) the production of a TCR comprising the α and β chain variable domains of the ILA TCR wherein one or both of the α and β chain variable domains comprise a mutation(s) in one or more of the amino acids identified in claims 7 and 8;
(b) contacting said mutated TCR with ILAKFLHWL-HLA-A*0201 under conditions suitable to allow the binding of the TCR to ILAKFLHWL-HLA-A*0201;
   and measuring the K_{D} and/or k_{off} of the interaction.

Preferred features of each aspect of the invention are as for each of the other aspects mutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

FIGS. 1a and 1b details the alpha chain variable domain amino acid and beta chain variable domain amino acid sequences of the native ILA TCR respectively.

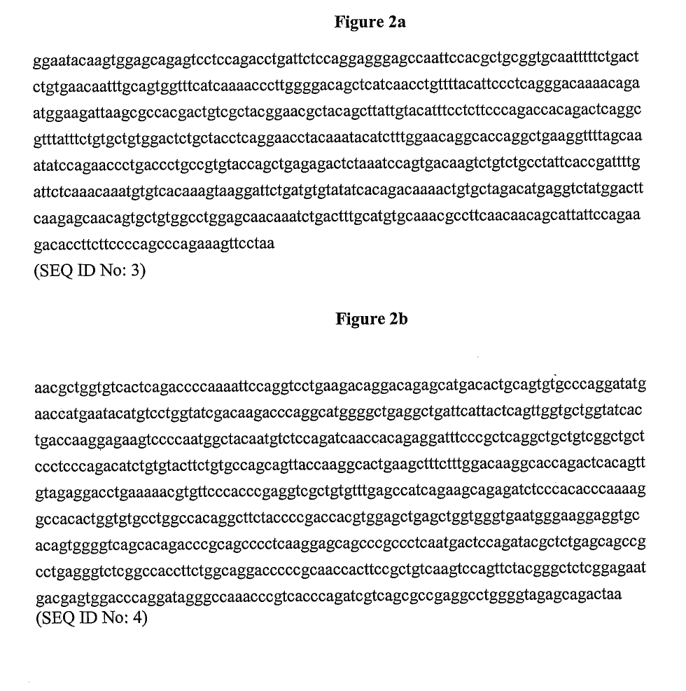

FIGS. 2a and 2b show respectively the DNA sequence of soluble versions of the native ILA TCR α and β chains.

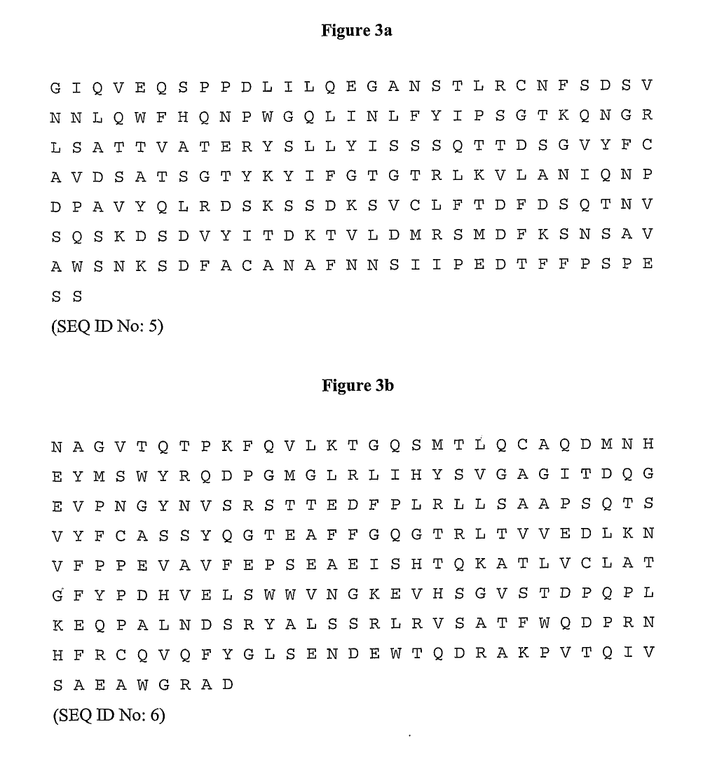

FIGS. 3a and 3b show respectively the ILA TCR α and β chain extracellular amino acid sequences produced from the DNA sequences of FIGS. 2a and 2b.

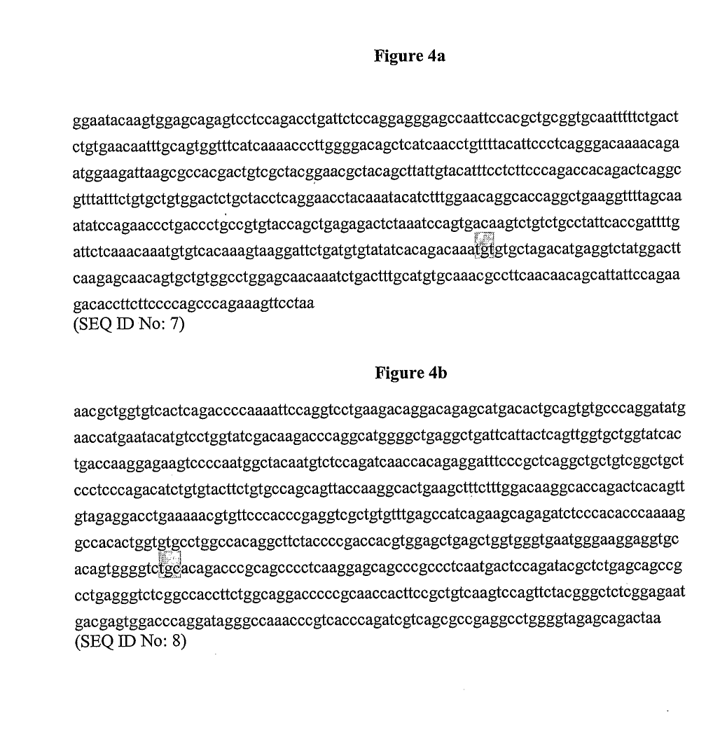

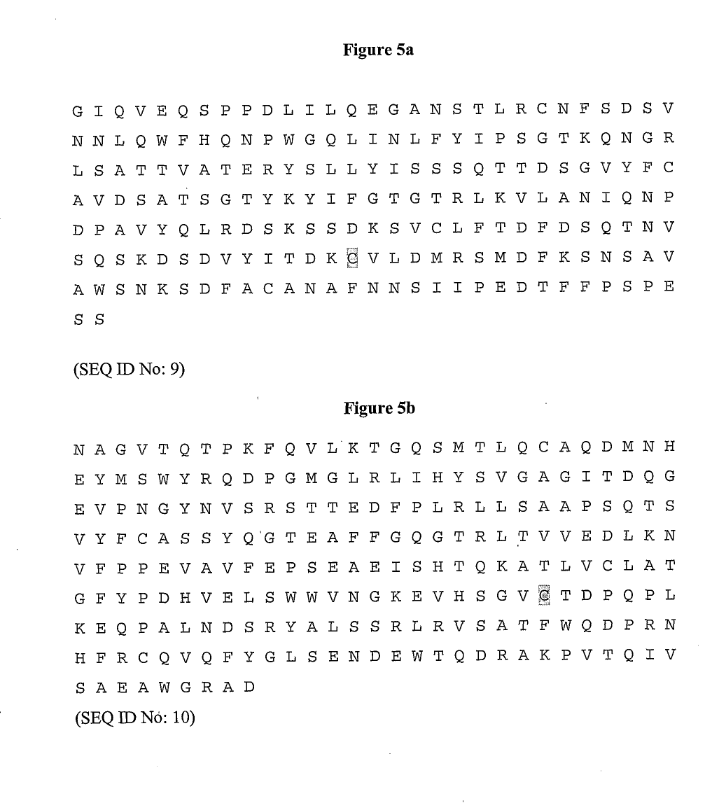

FIGS. 4a and 4b show respectively the DNA sequence of soluble versions of the ILA TCR α and β chains mutated to include additional cysteine residues to form a non-native disulphide bond. The mutated codon is indicated by shading.

FIGS. 5a and 5b show respectively the ILA TCR α and β chain extracellular amino acid sequences produced from the DNA sequences of FIGS. 4a and 4b. The introduced cysteine is indicated by shading.

FIG. 6 details the alpha chain variable domain amino acid sequences of the high affinity ILA TCR variants.

FIG. 7 details the beta chain variable domain amino acid sequences of the high affinity ILA TCR variants.

FIG. 8a details the amino acid sequence of a soluble form of TRAC.

FIG. 8b details the amino acid sequence of a soluble form of TRBC1. The amino acids which differ from those in TRBC2 are highlighted.

FIG. 8c details the amino acid sequence of a soluble form of TRBC2. The amino acids which differ from those in TRBC1 are highlighted.

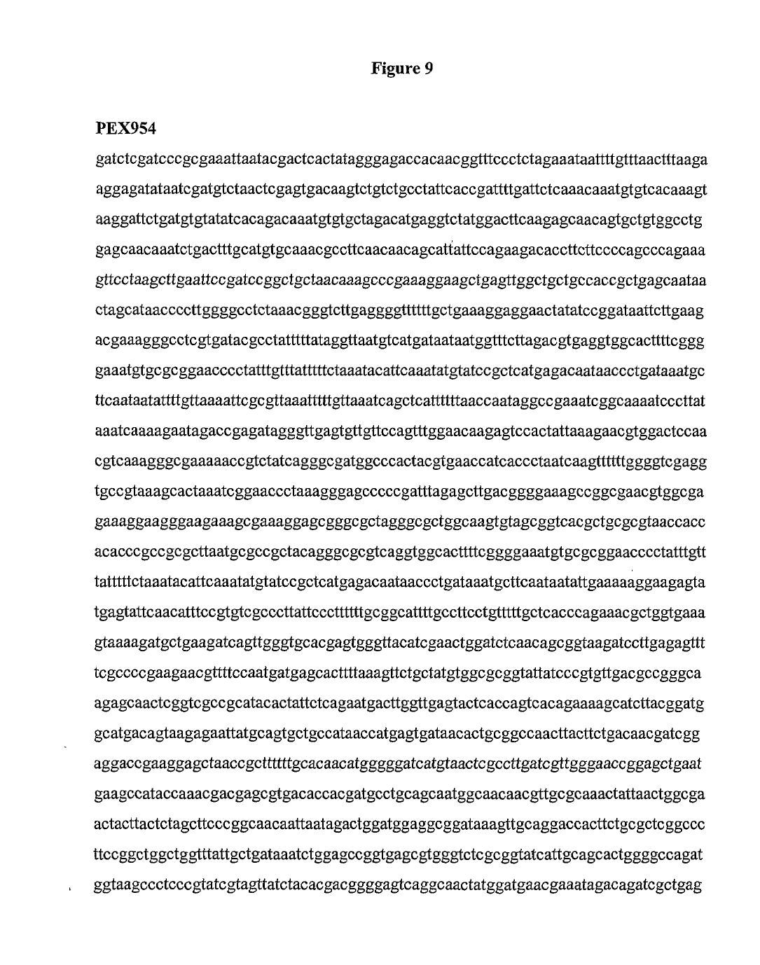

FIG. 9 details the DNA sequence of the pEX954 plasmid.

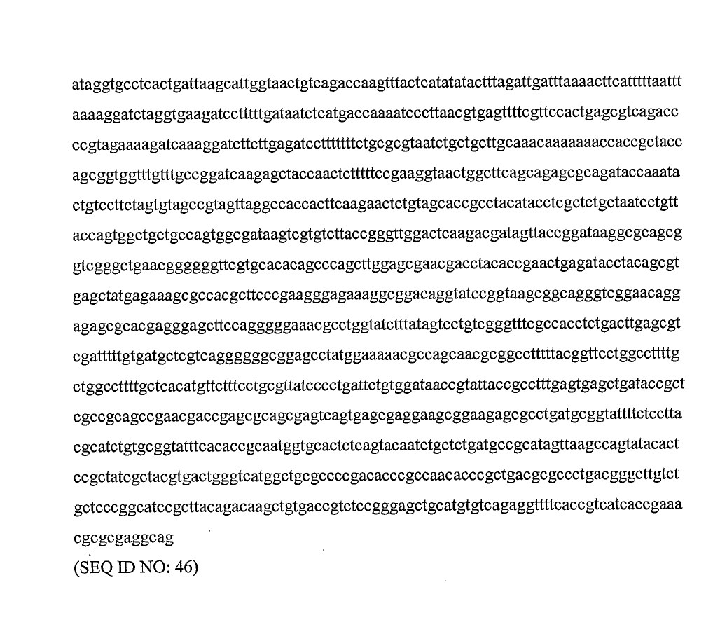

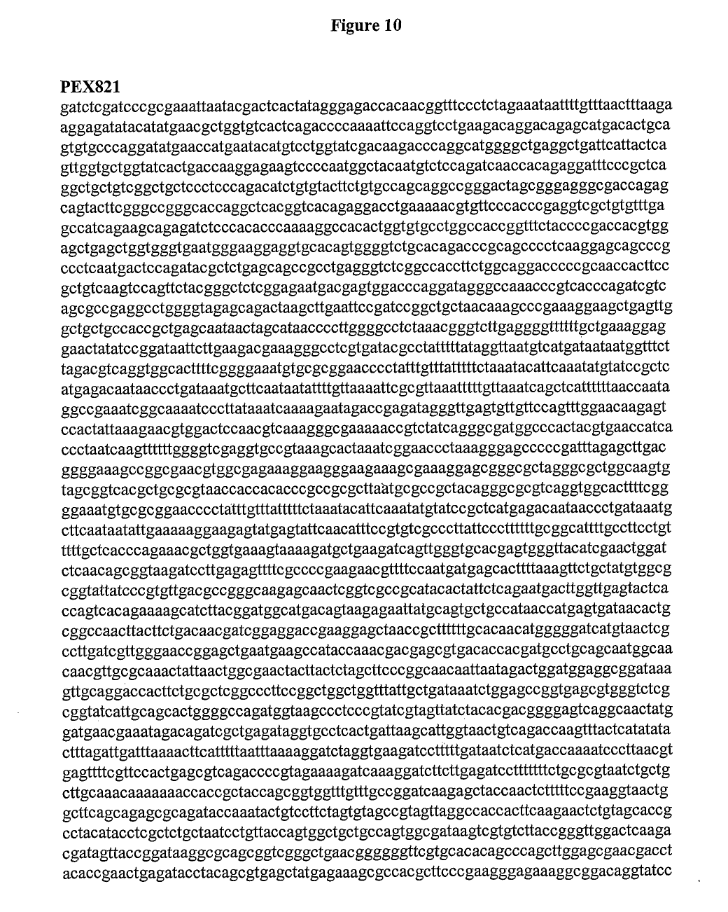

FIG. 10 details the DNA sequence of the pEX821 plasmid.

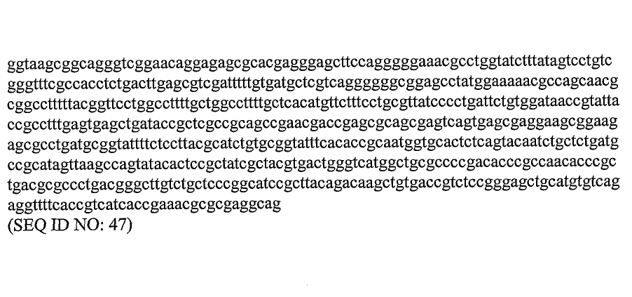

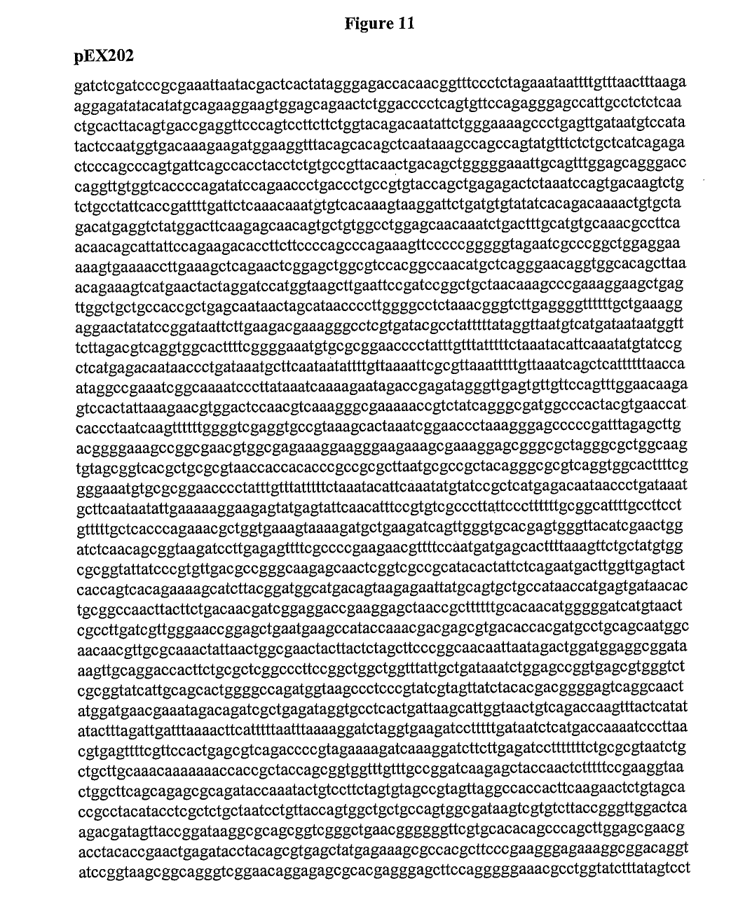

FIG. 11 details the DNA sequence of the pEX202 plasmid.

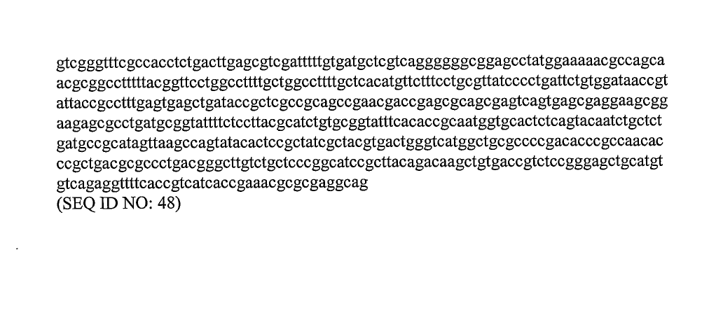

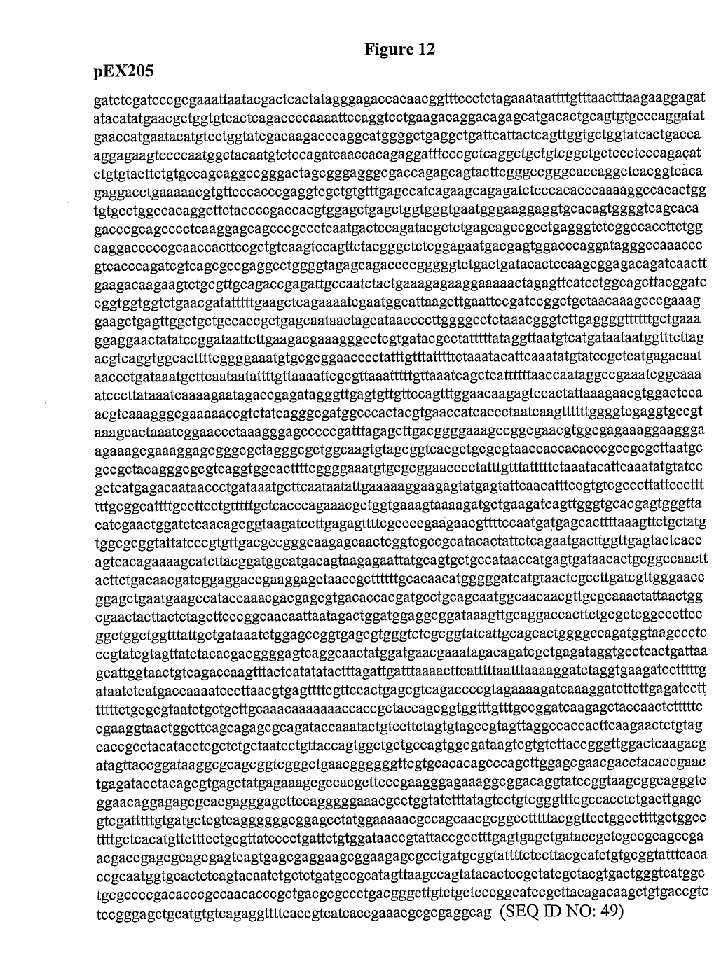

FIG. 12 details the DNA sequence of the pEX205 plasmid.

FIG. 13 details the alpha chain variable domain amino acid sequences of additional high affinity ILA TCR variants.

FIG. 14 details the beta chain variable domain amino acid sequences of additional high affinity ILA TCR variants.

FIG. 15a details the amino acid sequence of the alpha chain of the c13c1 ILA TCR.

FIG. 15b details the amino acid sequence of the beta chain of the c13c1 ILA TCR fused via a peptide linker to wild-type human IL-2. The mutations conferring high pMHC affinity to the TCR are underlined. The introduced cysteine residue in the TCR constant domain is highlighted and the linker and IL-2 sequence is in italics.

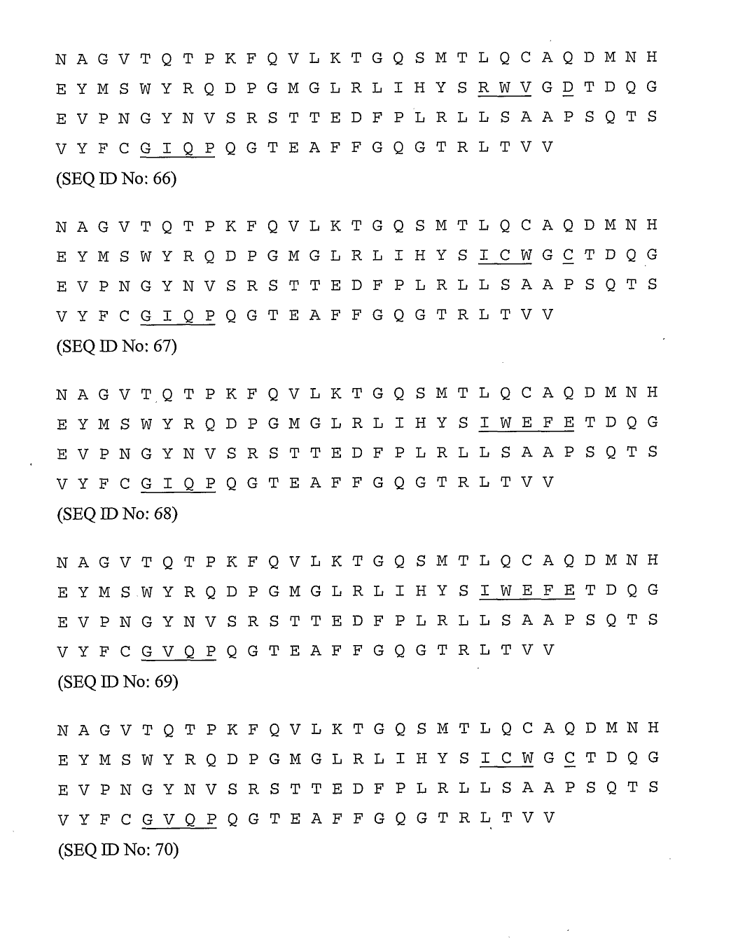

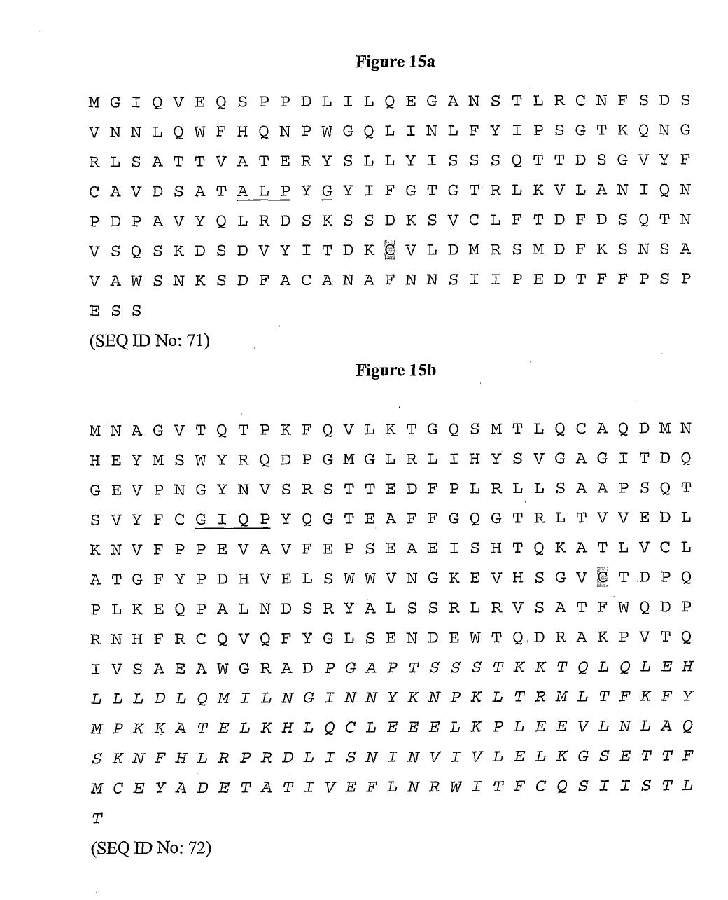

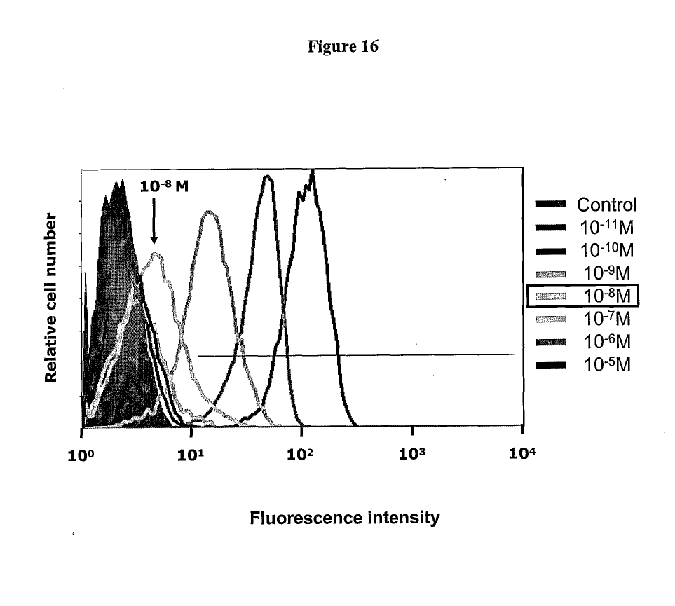

FIG. 16 shows FACs staining of T2 cell pulsed with a range of ILAKFLHWL peptide concentrations.

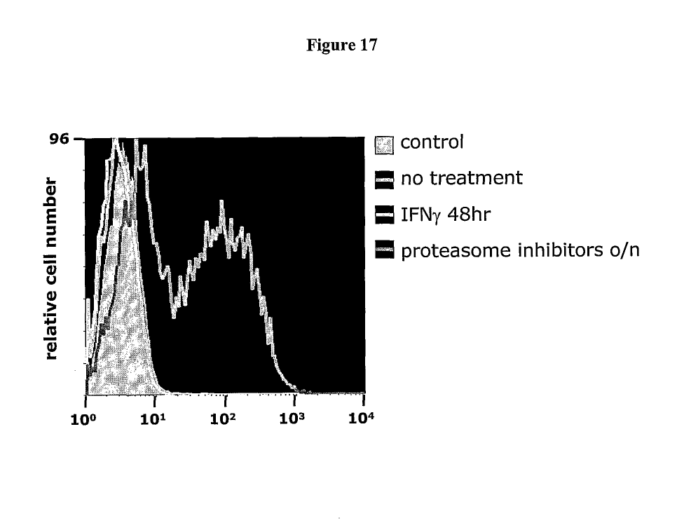

FIG. 17 shows FACs staining of J82 cells transfected with an ILAKFLHWL-producing ubiquitin minigene (±proteosome inhibitors)

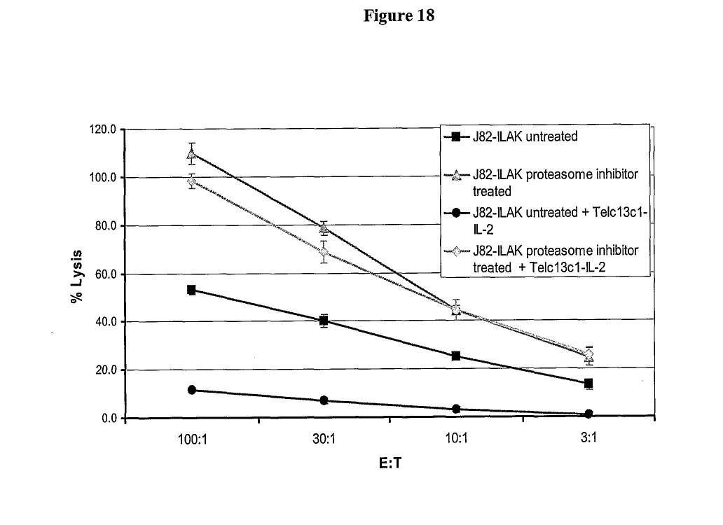

FIG. 18 shows inhibition of ILA-1 T cell clone-mediated killing of J82 cells transfected with an ILAKFLHWL-producing ubiquitin minigene by soluble high affinity c13c1 ILA TCR-IL-2 fusion protein.

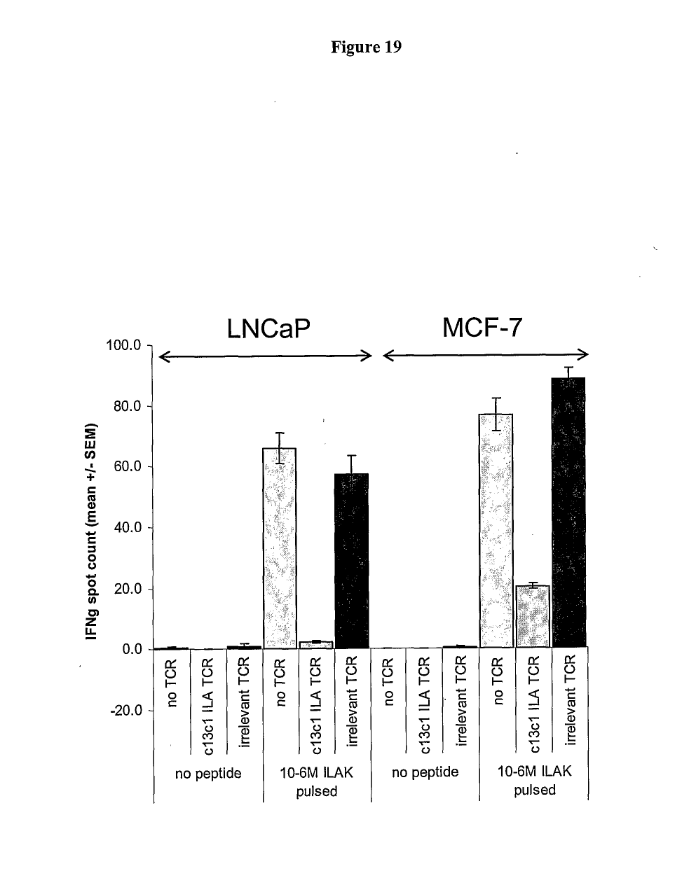

FIG. 19 shows ELISPOT data demonstrating the inhibition of T cell activation against peptide-pulsed LNCaP and MCF-7 target cells by soluble high affinity c13c1 ILA TCR-IL-2 fusion protein.

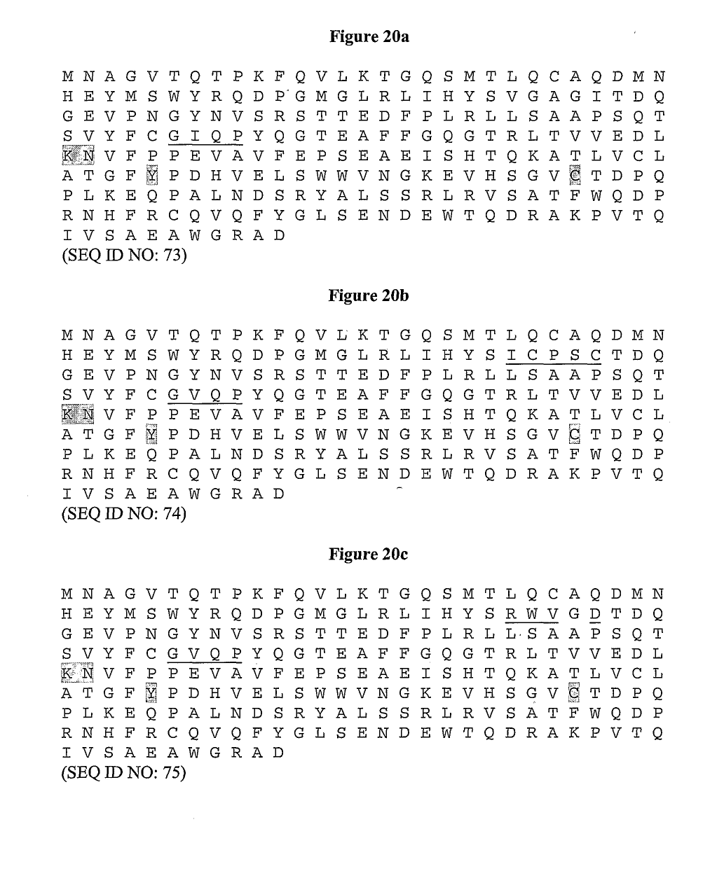

FIG. 20a shows the TRBC2 form of the beta chain of the high affinity ILA TCR c13c1. (SEQ ID NO: 73) The amino acids which differ from those in TRBC1 are highlighted.

FIG. 20b shows the TRBC2 form of the beta chain of the high affinity ILA TCR c13c1. (SEQ ID NO: 74) The amino acids which differ from those in TRBC1 are highlighted.

FIG. 20c shows the TRBC2 form of the beta chain of the high affinity ILA TCR c13c1. (SEQ ID NO: 75) The amino acids which differ from those in TRBC1 are highlighted.

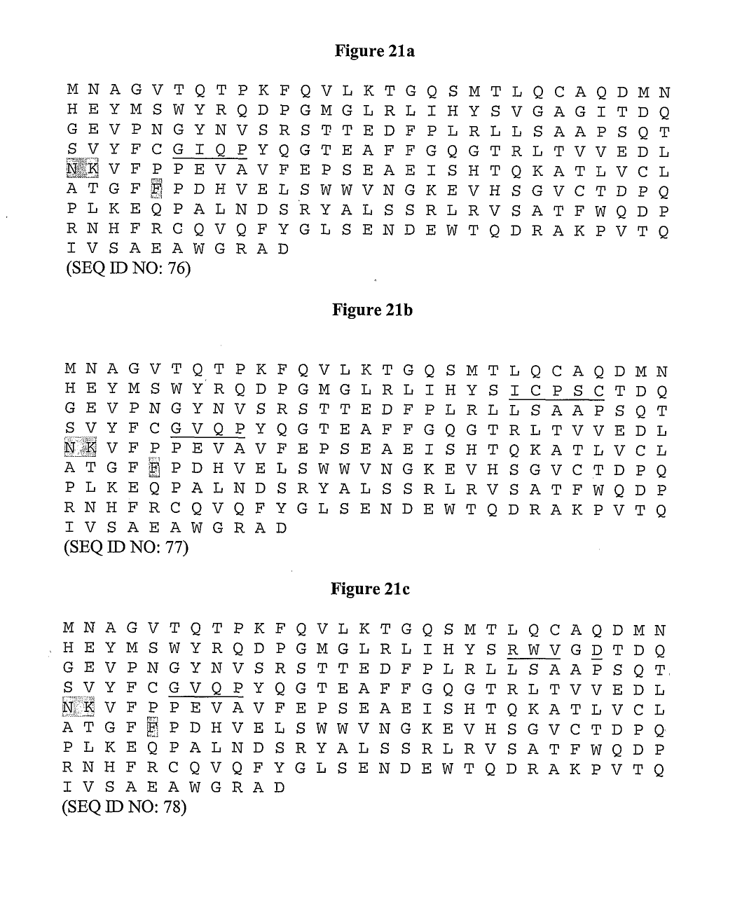

FIG. 21a shows the TRBC1 form of the beta chain of the high affinity ILA TCR c13c1. (SEQ ID NO: 76) The amino acids which differ from those in TRBC2 are highlighted.

FIG. 21b shows the TRBC1 form of the beta chain of the high affinity ILA TCR c13c1. (SEQ ID NO: 77) The amino acids which differ from those in TRBC2 are highlighted.

FIG. 21c shows the TRBC1 form of the beta chain of the high affinity ILA TCR c13c1. (SEQ ID NO: 78) The amino acids which differ from those in TRBC2 are highlighted.

### Example 1

### Production of a Soluble Disulfide-Linked TCR Comprising the Native ILA TCR Variable Domain

### RNA Isolation

Total RNA was isolated from 10000 clonal T cells by re-suspension in 100 μl tri-reagent (Sigma) and processing of the lysate according to the manufacturer's instructions. After the final precipitation the RNA was re-dissolved in 12.5 μl RNAse free water.

cDNA Production

To the above sample of RNA, 2.5 μl of 10 mM oligo-dT¹⁵ (Promega) was added and the sample incubated at 60° C. for 2 minutes then placed on ice. Reverse transcription was carried out using OmniscriptRT kit (Qiagen) by addition of 2 μl RT buffer (10×), 2 μl 5 mM dNTP, 1 μl Omniscript reverse transcriptase. The sample was mixed and incubated for 1 hour at 37° C. cDNA was then stored at −80° C.

The above cDNA was used as template. A panel of forward primers covering all possible alpha and beta variable chains was used to screen for, and amplify by PCR, alpha and beta chains genes. Primer sequences used for TCR chain gene amplification were designed from the NCBI website (http://www.ncbi.nlm.nih.gov/Entrez/) using accession numbers obtained from the T cell receptor Factsbook, (2001) LeFranc and LeFranc, Academic Press, ISBN 0-12-441352-8. Alpha-chain forward primers were designed to contain a ClaI restriction site and the universal alpha chain reverse primer a SalI restriction site. Beta-chain forward primers were designed to contain a AseI restriction site and universal beta reverse primer an AgeI restriction site.

Recipient vectors for the TCR gene fragments were based on a pGMT7 parent plasmid, which contains the T7 promoter for high level expression in E. coli strain BL21-DE3(pLysS) (Pan et al., Biotechniques (2000) 29 (6): 1234-8)

Alpha chain purified PCR products were digested with ClaI and SalII and ligated into pEX954 (see FIG. 9) cut with ClaI and XhoI.

Beta chain purified PCR products were digested with AseI and AgeI and ligated into pEX821 (See FIG. 10) cut with NdeI/AgeI.

### Ligation

The cut PCR product and cut vector were ligated using a rapid DNA ligation kit (Roche) following the manufacturers instructions.

Ligated plasmids were transformed into competent E. coli strain XL1-blue cells and plated out on LB/agar plates containing 100 mg/ml ampicillin. Following incubation overnight at 37° C., single colonies were picked and grown in 10 ml LB containing 100 mg/ml ampicillin overnight at 37° C. with shaking. Cloned plasmids were purified using a Miniprep kit (Qiagen) and the insert was sequenced using an automated DNA sequencer (Lark Technologies).

FIGS. 4a and 4b show respectively the DNA sequence of soluble versions of the ILA TCR α and β chains mutated to include additional cysteine residues to form a non-native disulphide bond.

FIGS. 5a and 5b show respectively the ILA TCR α and β chain extracellular amino acid sequences produced from the DNA sequences of FIGS. 4a and 4b

### Example 2

### Production of High Affinity Variants of the Soluble Disulfide Linked ILA TCR

The soluble disulfide-linked native ILA TCR produced as described in Example 1 can be used a template from which to produce the TCRs of the invention which have an increased affinity for the ILAKFLHWL (SEQ ID NO: 35)-HLA-A*0201 complex.

The amino sequences of the mutated TCR alpha and beta chain variable domains which demonstrate high affinity for the ILAKFLHWL-HLA-A*0201 complex are listed in FIGS. 6 and 7 respectively. (SEQ ID Nos: 11-22 and 23-31 respectively) As is known to those skilled in the art the necessary codon changes required to produce these mutated chains can be introduced into the DNA encoding these chains by site-directed mutagenesis. (QuickChange™ Site-Directed Mutagenesis Kit from Stratagene)

Briefly, this is achieved by using primers that incorporate the desired codon change(s) and the plasmids containing the relevant TCR chain as a template for the mutagenesis:

Mutagenesis was carried out using the following conditions: 50 ng plasmid template, 1 μl of 10 mM dNTP, 5 μl of 10× Pfu DNA polymerase buffer as supplied by the manufacturer, 25 pmol of fwd primer, 25 pmol of rev primer, 1 μl pfu DNA polymerase in total volume 50 μl. After an initial denaturation step of 2 mins at 95 C, the reaction was subjected to 25 cycles of denaturation (95 C, 10 secs), annealing (55 C 10 secs), and elongation (72 C, 8 mins). The resulting product was digested with DpnI restriction enzyme to remove the template plasmid and transformed into E. coli strain XL1-blue. Mutagenesis was verified by sequencing.

### Example 3

### Production of Soluble “Zippered” High Affinity TCRs

Alpha Chain—c-jun Leucine Zipper

The construct was made by PCR stitching.

For the 5′-end of the gene the plasmid coding for the high affinity TCR alpha chains and containing the code for the introduced inter-chain di-sulfide bridge was used as template. PCR with the following two primer pairs generated the desired variable domain.

5′-TRAV22 fwd

(SEQ ID NO: 38)

tctctcattaatgGGAATACAAGTGGAGCAGAGTCCT
C-alpha rev

(SEQ ID NO: 39)

CGGCAGGGTCAGGGTTCTGG

For the 3′-end of the gene the plasmid pEX202 (see FIG. 11), coding for a wild type affinity TCR alpha chain fused to human c-jun leucine zipper domain and not containing the code for the introduced inter-chain di-sulfide bridge, was used as template. PCR with the following primer pair generated the desired constant domain.

(SEQ ID NO: 40)

| | |
|---|---|
| C-alpha fwd | CCAGAACCCTGACCCTGCCG |

(SEQ ID NO: 41)

| | |
|---|---|
| 3′-alpha rev | aagcttcccgggggaactttctgggctggg |

The two products were mixed and diluted 1000 fold and 1 μl was used as template in a 50 μl PCR with 5′-TRAV21 fwd and 3′-alpha rev primers.

The resulting PCR product was digested using restriction enzymes AseI and XmaI and ligated into pEX202 cut with NdeI and XmaI.

PCRs were carried out using the following conditions: 50 pg plasmid template, 1 μl of 10 mM dNTP, 5 μl of 10×Pfu DNA polymerase buffer as supplied by the manufacturer, 25 pmol of fwd primer, 25 pmol of rev primer, 1 μl Pfu DNA polymerase in total volume 50 μl. After an initial denaturation step of 2 mires at 95 C, the reaction was subjected to 30 cycles of denaturation (95 C, 10 secs), annealing (55 C 10 secs), and elongation (72 C, 2 mins).

Beta Chain—c-fos Leucine Zipper

The construct was made by PCR stitching.

For the 5′-end of the gene plasmids coding for the high affinity TCR beta chains and containing the introduced inter-chain di-sulfide bridge were used as template. PCR with the following two primers generated the desired variable domain gene fragment.

(SEQ ID NO: 42)

| | |
|---|---|
| TRBV6-5 fwd | tctctcattaatgaatgctggtgtcactcagacccc |

(SEQ ID NO: 43)

| | |
|---|---|
| C-beta rev | CTTCTGATGGCTCAAACACAGC |

For the 3′-end of the gene the plasmid pEX205 (see FIG. 12), coding for a wild type affinity TCR beta chain fused to the human c-fos leucine zipper domain and not containing the code for the introduced inter-chain di-sulfide bridge, was used as template. PCR with the following two primers generated the desired constant domain gene fragment.

(SEQ ID NO: 44)

| | |
|---|---|
| C-beta fwd | GCTGTGTTTGAGCCATCAGAAG |

(SEQ ID NO: 45)

| | |
|---|---|
| TRBC rev | aagcttcccggggtctgctctaccccaggc |

The two products were mixed and diluted 1000 fold and 1 μl was used as template in a 50 μl PCR with TRBV6-5 fwd and TRBC rev primers. PCRs were carried out as described above.

The resulting PCR product was digested using restriction enzymes AseI and XmaI and ligated into pEX205 (FIG. 12) cut with NdeI and XmaI.

### Example 4

### Expression, Refolding and Purification of Soluble TCR

The expression plasmids containing the mutated α-chain and β-chain respectively as prepared in Examples 1, 2 or 3 were transformed separately into E. coli strain BL21pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 4000 rpm in a Beckman J-6B. Cell pellets were re-suspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, re-suspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl, 0.5% Triton-X100, 200 mM NaCl, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 30 mg aliquots and frozen at −70° C. Inclusion body protein yield was quantitated by solubilising with 6M guanidine-HCl and measurement with a Bradford dye-binding assay (PerBio).

Approximately 30 mg of TCR β chain and 60 mg of TCR α chain solubilised inclusion bodies were thawed from frozen stocks, samples were then mixed and the mixture diluted into 15 ml of a guanidine solution (6 M Guanidine-hydrochloride, 10 mM Sodium Acetate, 10 mM EDTA), to ensure complete chain de-naturation. The guanidine solution containing fully reduced and denatured TCR chains was then injected into 1 litre of the following refolding buffer: 100 mM Tris pH 8.5, 400 mM L-Arginine, 2 mM EDTA, 5 mM reduced Glutathione, 0.5 mM oxidised Glutathione, 5M urea, 0.2 mM PMSF. The redox couple (2-mercaptoethylamine and cystamine (to final concentrations of 6.6 mM and 3.7 mM, respectively) were added approximately 5 minutes before addition of the denatured TCR chains. The solution was left for 5 hrs±15 minutes. The refolded TCR was dialysed in Spectrapor 1 membrane (Spectrum; Product No. 132670) against 10 L 10 mM Tris pH 8.1 at 5° C.±3° C. for 18-20 hours. After this time, the dialysis buffer was changed to fresh 10 mM Tris pH 8.1 (10 L) and dialysis was continued at 5° C.±3° C. for another 20-22 hours.

sTCR was separated from degradation products and impurities by loading the dialysed refold onto a POROS 50HQ anion exchange column and eluting bound protein with a gradient of 0-500 mM NaCl over 50 column volumes using an Akta purifier (Pharmacia). Peak fractions were stored at 4° C. and analysed by Coomassie-stained SDS-PAGE before being pooled and concentrated. Finally, the sTCR was purified and characterised using a Superdex 200HR gel filtration column pre-equilibrated in HBS-EP buffer (10 mM HEPES pH 7.4, 150 mM NaCl, 3.5 mM EDTA, 0.05% nonidet p40). The peak eluting at a relative molecular weight of approximately 50 kDa was pooled and concentrated prior to characterisation by BIAcore surface plasmon resonance analysis.

### Example 5

### BIAcore Surface Plasmon Resonance Characterisation of sTCR Binding to Specific pMHC

A surface plasmon resonance biosensor (BIAcore 3000™) was used to analyse the binding of a sTCR to its peptide-MHC ligand. This was facilitated by producing single pMHC complexes (described below) which were immobilised to a streptavidin-coated binding surface in a semi-oriented fashion, allowing efficient testing of the binding of a soluble T-cell receptor to up to four different pMHC (immobilised on separate flow cells) simultaneously. Manual injection of HLA complex allows the precise level of immobilised class I molecules to be manipulated easily.

Biotinylated class I HLA-A*0201 molecules were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). HLA-A*0201-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The MHC light-chain or β2-microglobulin was also expressed as inclusion bodies in E. coli from an appropriate construct, at a level of ˜500 mg/litre bacterial culture.

E. coli cells were lysed and inclusion bodies are purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2 m into 0.4 M L-Arginine-HCl, 100 mM Tris pH 8.1, 3.7 mM cystamine, mM cysteamine, 4 mg/ml of the ILAKFLHWL peptide required to be loaded by the HLA-A*0201 molecule, by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. Two changes of buffer were necessary to reduce the ionic strength of the solution sufficiently. The protein solution was then filtered through a 1.5 μM cellulose acetate filter and loaded onto a POROS 50HQ anion exchange column (8 ml bed volume). Protein was eluted with a linear 0-500 mM NaCl gradient. HLA-A*0201-peptide complex eluted at approximately 250 mM NaCl, and peak fractions were collected, a cocktail of protease inhibitors (Calbiochem) was added and the fractions were chilled on ice.

Biotinylation tagged pMHC molecules were buffer exchanged into 10 mM Tris pH 8.1, 5 mM NaCl using a Pharmacia fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture was then allowed to incubate at room temperature overnight.

The biotinylated pHLA-A*0201 molecules were purified using gel filtration chromatography. A Pharmacia Superdex 75 HR 10/30 column was pre-equilibrated with filtered PBS and 1 ml of the biotinylation reaction mixture was loaded and the column was developed with PBS at 0.5 ml/min. Biotinylated pHLA-A*0201 molecules eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pHLA-A*0201 molecules were stored frozen at −20° C. Streptavidin was immobilised by standard amine coupling methods.

Such immobilised complexes are capable of binding both T-cell receptors and the coreceptor CD8αα, both of which may be injected in the soluble phase. Specific binding of TCR is obtained even at low concentrations (at least 40 μg/ml), implying the TCR is relatively stable. The pMHC binding properties of sTCR are observed to be qualitatively and quantitatively similar if sTCR is used either in the soluble or immobilised phase. This is an important control for partial activity of soluble species and also suggests that biotinylated pMHC complexes are biologically as active as non-biotinylated complexes.

The interactions between ILA sTCR containing a novel inter-chain bond and its ligand/MHC complex or an irrelevant HLA-peptide combination, the production of which is described above, were analysed on a BIAcore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The probe flow cells were prepared by immobilising the individual HLA-peptide complexes in separate flow cells via binding between the biotin cross linked onto β2 m and streptavidin which have been chemically cross linked to the activated surface of the flow cells. The assay was then performed by passing sTCR over the surfaces of the different flow cells at a constant flow rate, measuring the SPR response in doing so.

### To Measure Equilibrium Binding Constant

Serial dilutions of WT ILA sTCR were prepared and injected at constant flow rate of 5 μl min-1 over two different flow cells; one coated with ˜1000 RU of specific ILAKFLHWL-HLA-A*0201 complex, the second coated with ˜1000 RU of non-specific HLA-A2-peptide complex. Response was normalised for each concentration using the measurement from the control cell. Normalised data response was plotted versus concentration of TCR sample and fitted to a hyperbola in order to calculate the equilibrium binding constant, K_{D}. (Price & Dwek, Principles and Problems in Physical Chemistry for Biochemists (2^{nd} Edition) 1979, Clarendon Press, Oxford).

### To Measure Kinetic Parameters

For high affinity TCRs K_{D} was determined by experimentally measuring the dissociation rate constant, kd, and the association rate constant, ka. The equilibrium constant K_{D} was calculated as kd/ka.

TCR was injected over two different cells one coated with ˜300 RU of specific ILAKFLHWL-HLA-A*0201 complex, the second coated with ˜300 RU of non-specific HLA-A2-peptide complex. Flow rate was set at 50 μl/min. Typically 250 μl of TCR at ˜3 μM concentration was injected. Buffer was then flowed over until the response had returned to baseline. Kinetic parameters were calculated using Biaevaluation software. The dissociation phase was also fitted to a single exponential decay equation enabling calculation of half-life.

### Results

The interaction between a soluble disulfide-linked native ILA TCR and the ILAKFLHWL-HLA-A*0201 complex was analysed using the above methods and demonstrated a K_{D} of approximately 25 μM, an off-rate (k_{off}) of 1.1×10⁻¹ S⁻¹ and a half-life of 0.18 minutes.

The TCRs specified in the following table have a K_{D} of less than or equal to 1 μM and/or a k_{off} of 1×10⁻³ S⁻¹ or slower.

| Alpha chain variable | Beta chain variable |
|---|---|
| domain sequence, | domain sequence, |
| SEQ ID NO: | SEQ ID NO: |
| 1 | 23 |
| 1 | 24 |
| 1 | 25 |
| 1 | 26 |
| 1 | 27 |
| 1 | 28 |
| 1 | 29 |
| 1 | 30 |
| 1 | 31 |
| 11 | 2 |
| 12 | 2 |
| 13 | 2 |
| 14 | 2 |
| 15 | 2 |
| 16 | 2 |
| 17 | 2 |
| 18 | 2 |
| 19 | 2 |
| 20 | 2 |
| 21 | 2 |
| 22 | 2 |
| 50 | 2 |
| 51 | 2 |
| 52 | 2 |
| 53 | 2 |
| 54 | 2 |
| 55 | 2 |
| 56 | 2 |
| 57 | 2 |
| 58 | 2 |
| 59 | 2 |
| 60 | 2 |
| 17 | 25 |
| 17 | 23 |
| 12 | 23 |
| 16 | 23 |
| 14 | 23 |
| 12 | 25 |
| 15 | 23 |
| 19 | 23 |
| 20 | 23 |
| 11 | 61 |
| 11 | 62 |
| 11 | 63 |
| 11 | 64 |
| 11 | 65 |
| 14 | 65 |
| 16 | 66 |
| 22 | 65 |
| 20 | 31 |
| 17 | 31 |
| 17 | 66 |
| 18 | 65 |
| 19 | 65 |
| 17 | 65 |
| 21 | 65 |
| 11 | 31 |
| 19 | 31 |
| 16 | 31 |
| 12 | 31 |
| 11 | 66 |
| 17 | 67 |
| 11 | 67 |
| 15 | 66 |
| 15 | 68 |
| 11 | 67 |
| 15 | 8 |
| 11 | 69 |
| 15 | 70 |
| 15 | 69 |
| 17 | 69 |
| 11 | 70 |
| 17 | 70 |
| 11 | 28 |
| 11 | 23 |

### Example 6

### Competition ELISA Analysis of the Binding of Native and Mutated Disulphide-Linked ILA TCRS Displayed on Phage Particles

The following competition ELISA assay using native and mutated disulfide-linked ILA TCRs displayed on the phage particles was used to assess the affinity of these molecules for the ILAKFLHWL-HLA-A*0201 complex:

Binding of the ILA TCR-displaying phage particles to immobilised ILAKFLHWL-HLA-A*0201 complex in ELISA was detected with primary rabbit anti-fd antisera (Sigma) followed by alkaline phosphatase (AP) conjugated anti-Rabbit mAb (Sigma) in the presence of varying amounts of soluble ILAKFLHWL-HLA-A*0201 complex. Non-specific protein binding sites in the plates can be blocked with 2% MPBS or 3% BSA-PBS.

### Materials and Reagents

1. Coating buffer, PBS
2. PBS: 138 mM NaCl, 2.7 mM KCl, 10 mM Na₂HPO₄, 2 mM KH₂PO₄
3. MPBS, 3% marvel-PBS
4. PBS-Tween: PBS, 0.1% Tween-20
5. Substrate solution, Sigma FAST pNPP, Cat# N2770

### Method

1. Rinse NeutrAvidin coated wells twice with PBS.
2. Add 25 μl of biotin-HLA-A2 Tax or biotin-HLA-A2 NYESO in PBS at concentration of 10 μg/ml, and incubate at room temperature for 30 to 60 min.
3. Rinse the wells twice with PBS
4. Add 300 μl of 3% Marvel-PBS, and incubate at room temperature for 1 hr. Mix the TCR-phage suspension with 1 volume of 3% Marvel-PBS and incubate at room temperature.
5. Rinse the wells twice with PBS
6. Add 25 μl of the mixture of phage-A6 TCR/Marvel-PBS and the required amount (20 nM or 200 nM) of soluble ILAKFLHWL-HLA-A*0201 complex, incubate on ice for 1 hr
7. Rinse the wells three times with ice-cold PBStween, and three times with ice-cold PBS.
8. Add 25 μl of ice cold rabbit anti-fd antibody diluted 1:1000 in Marvel-PBS, and incubate on ice for 1 hr
9. Rinse the wells three times with ice-cold PBStween, and three times with ice-cold PBS.
10. Add 25 μl of ice cold anti-rabbit mAb-Ap conjugate diluted 1:50,000 in Marvel-PBS, and incubate on ice for 1 hr
11. Rinse the wells three times with ice-cold PBStween, and three times with ice-cold PBS.
12. Add 150 μl of Alkaline phosphatase yellow to each well and read the signal at 405 nm.

The degree of inhibition of binding that occurs for a given addition of soluble ILAKFLHWL-HLA-A*0201 is proportional to the affinity of the TCR for the ILAKFLHWL-HLA-A*0201 complex.

### Results

| | | | % Binding |
|---|---|---|---|
| | | % Binding | Inhibition |
| α Variable | β Variable | Inhibition | 200 nM Sol. |
| Domain | Domain | 20 nM Sol. pMHC | pMHC |
| SEQ ID NO: 1 | SEQ ID NO: 2 | 0 | 0 |
| SEQ ID NO: 1 | SEQ ID NO: 23 | 70 | 95 |
| SEQ ID NO: 1 | SEQ ID NO: 24 | 64 | 89 |
| SEQ ID NO: 1 | SEQ ID NO: 25 | 88 | 93 |
| SEQ ID NO: 1 | SEQ ID NO: 26 | 84 | 96 |
| SEQ ID NO: 1 | SEQ ID NO: 27 | 68 | 96 |
| SEQ ID NO: 1 | SEQ ID NO: 28 | 88 | 94 |
| SEQ ID NO: 1 | SEQ ID NO: 29 | 79 | 92 |
| SEQ ID NO: 1 | SEQ ID NO: 30 | 79 | 95 |
| SEQ ID NO: 1 | SEQ ID NO: 31 | 33/80 | 95 |
| SEQ ID NO: 11 | SEQ ID NO: 2 | 82 | 98 |
| SEQ ID NO: 12 | SEQ ID NO: 2 | 87 | 98 |
| SEQ ID NO: 13 | SEQ ID NO: 2 | 77 | 97 |
| SEQ ID NO: 14 | SEQ ID NO: 2 | 88 | 98 |
| SEQ ID NO: 15 | SEQ ID NO: 2 | 47 | 92 |
| SEQ ID NO: 16 | SEQ ID NO: 2 | 67 | 95 |
| SEQ ID NO: 17 | SEQ ID NO: 2 | 63 | 95 |
| SEQ ID NO: 18 | SEQ ID NO: 2 | 47 | 94 |
| SEQ ID NO: 19 | SEQ ID NO: 2 | 61 | 96 |
| SEQ ID NO: 20 | SEQ ID NO: 2 | 80 | 95 |
| SEQ ID NO: 21 | SEQ ID NO: 2 | 71 | 97 |
| SEQ ID NO: 22 | SEQ ID NO: 2 | 79 | 95 |

### Example 7

### In-vitro Cell Staining Using High Affinity Clone c13c1 ILA TCR—IL-2 Fusion Protein

T2 lymphoblastoid cells were pulsed with a telomerase-derived ILAKFLHWL (540-548) peptide at a range of concentrations (10⁻⁵-10⁻¹¹ M) for 180 minutes at 37° C. After pulsing, cells were washed in serum-free RPMI and 5×10⁵ cells were incubated with high affinity clone c13c1 ILA TCR/IL-2 fusion protein for 10 min at room temperature, followed by secondary anti-IL-2 mAb conjugated with PE (Serotec) for 15 min at room temperature. After washing, bound TCR-IL-2 was quantified by flow cytometry using a FACSVantage SE (Becton Dickinson).Controls, also using peptide-pulsed T2 cells were included where TCR-IL-2 was omitted.

FIG. 15a details the amino acid sequence of the alpha chain of the c13c1 ILA TCR. (SEQ ID NO: 71) FIG. 15b details the amino acid sequence of the beta chain of the c13c1 ILA TCR fused via a peptide linker to wild-type human IL-2. (SEQ ID NO: 72) The alpha and beta chain variable domain mutations contained within the soluble c13c1 ILA TCR—IL-2 fusion protein correspond to those detailed in SEQ ID NO: 11 and SEQ ID NO: 23 respectively. Note that SEQ ID NOs: 71 and 72 have been provided in a form which includes the N-terminal methionine (M).

In similar experiments, J82 bladder cancer cells transfected with a telomerase-derived ILAKFLHWL (540-548) peptide expressing ubiquitin minigene construct were used. The J82 cells were transfected using substantially the methods described in (Rimoldi et al., (2000) J. Immunol. 165 7253-7261). Cells were labelled as described above.

FIG. 16 shows FACs staining of T2 cell pulsed with a range of ILAKFLHWL peptide concentrations.

FIG. 17 shows FACs staining of J82 cells transfected with an ILAKFLHWL-producing ubiquitin minigene (±proteosome inhibitors).

### Example 8

### Soluble TCR-Mediated Inhibition of CTL Activation Using High Affinity c13c1 ILA TCR-IL-2 Fusion Protein

The following assay was carried out to demonstrate that the soluble high affinity c1c13 ILA-IL-2 TCR was capable of inhibiting cell lysis mediated by an ILAKFLHWL-HLA-A*0201 specific CTL clone (ILA-1).

### In-vitro Cell Lysis Assay

J82 cells transfected with an ILAKFLHWL encoding mini-gene construct were treated with, or without, proteosomes inhibitors (1.5 μM epoxomicin and 20 μM calpain inhibitor I) for 16 hours before the CTL assay.

These target cells were treated with trypsin for 5 minutes at 37° C. The cells are then washed and re-suspended in R10 media with 3 μl BATDA reagent/1×10⁶ cells as directed by the instructions supplied with the Europium/DELFIA assay kit. (Perkin Elmer) These cells are then incubated for 30 minutes at 37° C., prior to washing three times in R10 media supplemented with 1 mM sulfinpyrazone.

5000 target cells were then plated out per well in 50 μl of R10 media in a 96 well plate (Nunc).

The following was then added to the above target cell cultures:

1×10⁻⁷ M high affinity c13c1 ILA TCR-IL-2 fusion protein in 50 μl of R10 media.

ILAKFLHWL-HLA-A*0201 specific T cell clone (ILA-1) at a range of cell numbers in 50 μl of R10 media. (To give Effector: Target ratios of 100:1, 30:1, 10:1 and 3:1)

These cultures were then incubated for 2 hours at 37° C., 5% CO₂. 20 μl of supernatant was then removed from each well and placed into a black opaque 96 well plate (Nunc). 180 μl of Europium solution from the Europium/DELFIA assay kit was then added to each well and the level of target cell lysis that had occurred was assayed by time-resolved fluorescence in a Wallac Victor 2. (Perkin Elmer)

### Calculations:

% cell lysis=100×(RFU_{Exp}−RFU_{Spont}/(RFU_{Max}−RFU_{Spont})

### Wherein:

RFU—is relative fluorescence units
RFU_{Exp}—is the RFU measured in the sample wells—cell free background RFU.
RFU_{Spont}—is the RFU measured in the sample wells not containing any Effector cells—cell free background RFU.
RFU_{Max}—is the RFU measured in the sample wells to which triton x-100 was added—cell free background RFU.

### Results

The soluble high affinity c13c1 ILA TCR-IL-2 fusion protein caused a significant inhibition of ILA-1 T cell clone-mediated killing of J82 target cells in the absence of proteosomes inhibitor treatment. (See FIG. 18)

### Example 9

### CTL Activation ELISPOT Assay

The following assay was carried out to demonstrate that the soluble high affinity c1c13 ILA-IL-2 TCR was capable of inhibiting activation of an ILAKFLHWL-HLA-A*0201 specific CTL clone (ILA-1). IFN-γ production was used as the read-out for CTL activation.

### Reagents

R10 Assay media: 10% FCS (heat-inactivated, Gibco, cat# 10108-165), 88% RPMI 1640 (Gibco, cat# 42401-018), 1% glutamine (Gibco, cat# 25030-024) and 1% penicillin/streptomycin (Gibco, cat# 15070-063).

Peptide: (obtained from various sources) initially dissolved in DMSO (Sigma, cat# D2650) at 4 mg/ml and frozen, then made up to 200 μM in assay media and frozen in small aliquots. One aliquot of 200 μM peptide is thawed for each experiment so that the peptide has only gone through two freeze-thaw cycles.

Wash buffer: 0.01M PBS/0.05% Tween 20 (1 sachet of Phosphate buffered saline with Tween 20, pH7.4 from Sigma, Cat. # P-3563 dissolved in 1 litre distilled water gives final composition 0.01M PBS, 0.138M NaCl, 0.0027M KCl, 0.05% Tween 20). PBS (Gibco, cat#10010-015).

The EliSpot kit contains all other reagents required i.e. capture and detection antibodies, skimmed milk powder, BSA, streptavidin-alkaline phosphatase, BCIP/NBT solution (Human IFN-g PVDF Eli-spot 20×96 wells with plates (IDS cat# DC-856.051.020, DC-856.000.000—use both codes on an order). The following method is based on the instructions supplied with each kit but contains some alterations.

### Method

ELISPOT plates were prepared according to the manufacturers instructions. (Diaclone kit, Immunodiagnostic systems, UK

LNCaP or MCF-7 cancer cell line target cells were treated with trypsin for 5 minutes at 37° C. The cells are then washed and re-suspended in R10 media with or without 1 uM ILAKFLHWL peptide, then incubated for 1 hour at 37° C., 5% CO₂.

50,000 target cells per well were then plated out into a 96 well ELISPOT plate.

To these plates the following was added to the relevant well:

1×10-7 M of the high affinity c13c1 ILA TCR, or an irrelevant high affinity TCR in 50 μl of R10 media.

500 ILA-1 effector cells in 50 μl of R10 media.

The plates were then incubated for 5 hours at 37° C., 5% CO₂. The ELISPOT was then processed according to manufacturer's instructions.

### Results

The soluble high affinity c1c13 ILA TCR strongly inhibits the activation of ILA-1 T cell clones against both MCF-7 and LNCaP cancer cells, as measured by IFN-γ production. Whereas the irrelevant soluble high affinity TCR had no effect on the activation of this T cell clone. (See FIG. 19)

1. A T-cell receptor (TCR) having the property of binding to ILAKFLHWL (SEQ ID NO:37)-HLA-A*0201 and comprising at least one TCR α chain variable domain and/or at least one TCR β chain variable domain characterized in that said TCR has a K_{D} for the said ILAKFLHWL (SEQ ID NO:37)-HLA-A*0201 complex of less than or equal to 1 μM and/or has an off-rate (k_{off}) for the ILAKFLHWL (SEQ ID NO:37)-HLA-A*0201 complex of 1×10⁻³ S⁻¹ or slower.

2. A TCR as claimed in claim 1 comprising both an α chain variable domain and an TCR β chain variable domain.

3. A TCR as claimed in claim 1 which is an αα or ββ homodimer.

4. A T-cell receptor (TCR) as claimed in claim 1 wherein the said K_{D} and/or k_{off} is/are as measured by Surface Plasmon Resonance.

5. A TCR as claimed in claim 1 which is mutated relative to the native ILA TCR α chain variable domain (SEQ ID No: 1) and/or β chain variable domain (SEQ ID NO: 2) in at least one complementarity determining region.

6. A TCR as claimed in claim 1 which is mutated relative to the native ILA TCR α chain variable domain (SEQ ID No: 1) and/or β chain variable domain (SEQ ID NO: 2) in at least one variable domain framework region.

7. A TCR as claimed in claim 1 wherein one or more of alpha chain variable domain amino acids 94S, 95G, 96T, 97Y and 98K using the numbering shown in SEQ ID NO: 1 are mutated.

8. A TCR as claimed in claim 1 wherein one or more of beta chain variable domain amino acids 19M, 50V, 51G, 52A, 53G, 54I, 92A, 93S, 94S and 95Y using the numbering shown in SEQ ID NO: 2 are mutated.

9. A TCR as claimed in claim 1 comprising one or more of alpha chain variable domain amino acids 94A, 94L, 94Q, 94T, 94G, 94R, 94M, 94H, 94K, 94E, 94N, 95L, 95P, 95I, 95V, 95M, 96P, 96M, 97F 98G or 98A using the numbering shown in SEQ ID NO: 1.

10. A TCR as claimed in claim 1 comprising one or more of beta chain variable domain amino acids 19V, 50I, 50L, 50R, 51H, 51C, 51W, 52P, 52W, 52E, 52V, 53E, 53S, 53F, 54Y, 54V, 54C, 54E, 54D, 92G, 93I, 93L, 93V, 94Q and 95P using the numbering shown in SEQ ID NO: 2.

11. A TCR as claimed in claim 1 comprising one of the alpha chain variable domain amino acid sequences shown in (SEQ ID Nos: 11 to 22 or 50 to 60), optionally comprising one or more phenotypically silent substitutions.

12. A TCR as claimed in claim 1 comprising one of the beta chain variable domain amino acid sequences shown in (SEQ ID Nos: 23 to 31 or 61 to 70) optionally comprising one or more phenotypically silent substitutions.

13. (canceled)

14. A TCR as claimed in claim 2 comprising the alpha chain variable domain shown in the SEQ ID NO: 11 and the beta chain variable domain shown in the SEQ ID NO: 23, optionally comprising one or more phenotypically silent substitutions.

15. A TCR as claimed in claim 2 comprising the alpha chain variable domain shown in the SEQ ID NO: 11 and the beta chain variable domain shown in the SEQ ID NO: 64, optionally comprising one or more phenotypically silent substitutions.

16. A TCR as claimed in claim 2 comprising the alpha chain variable domain shown in the SEQ ID NO: 11 and the beta chain variable domain shown in the SEQ ID NO: 65, optionally comprising one or more phenotypically silent substitutions.

17. A TCR as claimed in claim 1 further comprising the alpha chain constant region amino acid sequence shown in SEQ ID NO: 32, and/or one of the beta chain amino acid constant region sequences shown in SEQ ID NOs: 33 and 34, optionally comprising one or more phenotypically silent substitutions.

18. A TCR as claimed in claim 1 which is a dimeric T cell receptor (dTCR) or a single chain T cell receptor (scTCR).

19. A TCR as claimed in claim 4 which is a scTCR comprising
a first segment constituted by an amino acid sequence corresponding to a TCR α chain variable domain
a second segment constituted by an amino acid sequence corresponding to a TCR β chain variable domain sequence fused to the N terminus of an amino acid sequence corresponding to a TCR β chain constant domain extracellular sequence, and
a linker sequence linking the C terminus of the first segment to the N terminus of the second segment,

20. A TCR as claimed in claim 4 which is a scTCR comprising
a first segment constituted by an amino acid sequence corresponding to a TCR β chain variable domain
a second segment constituted by an amino acid sequence corresponding to a TCR α chain variable domain sequence fused to the N terminus of an amino acid sequence corresponding to a TCR α chain constant domain extracellular sequence, and
a linker sequence linking the C terminus of the first segment to the N terminus of the second segment.

21. A TCR as claimed in claim 19 further comprising a disulfide bond between the first and second chains, said disulfide bond being one which has no equivalent in native αβT cell receptors, and wherein the length of the linker sequence and the position of the disulfide bond being such that the variable domain sequences of the first and second segments are mutually orientated substantially as in native αβ T cell receptors.

22. A scTCR as claimed in claim 19 wherein the linker sequence links the C terminus of the first segment to the N terminus of the second segment.

23. A scTCR as claimed in claim 19 wherein the linker sequence has the formula —PGGG-(SGGGG)₅-P— (SEQ ID NO: 35) or —PGGG-(SGGGG)₆-P— (SEQ ID NO: 36) wherein n is 5 or 6 and P is proline, G is glycine and S is serine.

24. A TCR as claimed in claim 1 which is an dTCR comprising
a first polypeptide wherein a sequence corresponding to a TCR α chain variable domain sequence is fused to the N terminus of a sequence corresponding to a TCR α chain constant domain extracellular sequence, and
a second polypeptide wherein a sequence corresponding to a TCR β chain variable domain sequence fused to the N terminus a sequence corresponding to a TCR β chain constant domain extracellular sequence,
the first and second polypeptides being linked by a disulfide bond which has no equivalent in native αβ T cell receptors.

25. A TCR as claimed in claim 24 wherein the disulfide bond links amino acid residues of the said constant domain sequences, which disulfide bond has no equivalent in native TCRs.

26. A TCR as claimed in claim 25 wherein the said disulfide bond is between cysteine residues corresponding to amino acid residues whose β carbon atoms are less than 0.6 nm apart in native TCRs.

27. A TCR as claimed in claim 25 wherein the said disulfide bond is between cysteine residues substituted for Thr 48 of exon 1 of TRAC*01 and Ser 57 of exon 1 of TRBC1*01 or TRBC2*01 or the non-human equivalent thereof.

28. A TCR as claimed in claim 18 wherein the dTCR or scTCR binding part includes a disulfide bond between residues corresponding to those linked by a disulfide bond in native TCRs.

29. A TCR as claimed in claim 14 wherein the dTCR or scTCR binding part does not contain a sequence corresponding to transmembrane or cytoplasmic sequences of native TCRs.

30. A soluble TCR consisting of the a pair of alpha chain and beta chain amino acid sequences selected from the group consisting of:
SEQ ID NO: 71 and SEQ ID NO: 73;
SEQ ID NO:71 and SEQ ID NO:76;
SEQ ID NO:71 and SEQ ID NO:74;
SEQ ID NO:71 and SEQ ID NO:77;
SEQ ID NO:71 and SEQ ID NO:75; and
SEQ ID NO:71 and SEQ ID NO:78.

31-35. (canceled)

36. The TCR of claim 30 wherein the TCR is associated with at least one polyalkylene glycol chain(s).

37. A TCR as claimed in claim 36 wherein the polyalkylene glycol chain(s) is/are covalently linked to the TCR.

38. A TCR as claimed in claim 26 wherein the polyalkylene glycol chain(s) comprise(s) at least two polyethylene glycol repeating units.

39. The TCR of claim 1 further comprising a reactive cysteine at the C terminal or N-terminal of the alpha or beta chains thereof.

40. The TCR of claim 1 associated with a therapeutic agent or detectable moiety.

41. A TCR as claimed in claim 40 wherein the TCR is covalently linked to a therapeutic agent or detectable moiety.

42. A TCR as claimed in claim 40 wherein the therapeutic agent or detectable moiety is covalently linked to the C terminus of one or both TCR chains.

43. The TCR of claim 40 associated with a therapeutic agent which is an immune effector molecule.

44. A TCR as claimed in claim 43 wherein the immune effector molecule is a cytokine.

45. A TCR as claimed in claim 43 wherein the immune effector molecule is IL-2, or a functional variant or fragment thereof.

46. The TCR of claim 40 associated with a therapeutic agent which is a cytotoxic agent.

47. The TCR of claim 40 associated with a therapeutic agent which is a radionuclide.

48. A multivalent TCR complex comprising at least two TCRs as claimed in claim 1.

49. multivalent TCR complex of claim 48 wherein the at least two TCRs are linked by a non-peptidic polymer chain or a peptidic linker sequence.

50. A TCR complex as claimed in claim 49 wherein the polymer chain or peptidic linker sequence extends between amino acid residues of each TCR which are not located in a variable region sequence of the TCR.

51. The TCR complex of claim 49 in which the TCRs are linked by a polyalkylene glycol chain or a peptidic linker derived from a human multimerisation domain.

52. A TCR complex as claimed in claim 51 wherein a divalent alkylene spacer radical is located between the polyalkylene glycol chain and its point of attachment to a TCR of the complex.

53. The TCR of claim 49 wherein the polyalkylene glycol chain comprises at least two polyethylene glycol repeating units.

54. A multivalent TCR complex comprising at least two TCRs as claimed in claim 1 wherein (i) at least one of said TCRs is associated with a therapeutic agent.

55. An isolated cell presenting the TCR of claim 1.

56. A pharmaceutical composition comprising the TCR of claim 1 a TCR together with a pharmaceutically acceptable carrier.

57-58. (canceled)

59. A method of producing a high affinity TCR having the property of binding to ILAKFLHWL (SEQ ID NO:37)-HLA-A*0201. CHARACTERISED IN THAT the TCR (i) comprises at least one TCR α chain variable domain and/or at least one TCR β chain variable domain and (ii) has a K_{D} for the said ILAKFLHWL (SEQ ID NO:37)-HLA-A*0201 complex of less than 1 μM and/or an off-rate (k_{off}) for the ILAKFLHWL (SEQ ID NO:37)-HLA-A*0201 complex of less than 1×10⁻³ said method comprising:
(a) the production of a TCR comprising the α and β chain variable domains of the ILA TCR wherein one or both of the α and β chain variable domains comprise a mutation(s) in one or more of the amino acids selected from the group consisting of 94S, 95G, 96T, 97Y and 98K of the α chain and 94A, 94L, 94Q, 94T, 94G, 94R, 94M, 94H, 94K, 94E, 94N, 95L, 95P, 95I, 95V, 95M, 96P, 96M, 97F 98G or 98A of the β chain;
(b) contacting said mutated TCR with ILAKFLHWL (SEQ ID NO:37)-HLA-A*0201 under conditions suitable to allow the binding of the TCR to ILAKFLHWL (SEQ ID NO:37)-HLA-A*0201;
and measuring the Kd and/or k_{off} of the interaction.

60. A pharmaceutical composition comprising a plurality of cells of claim 55 together with a pharmaceutically acceptable carrier.

61. A TCR as claimed in claim 2 comprising the alpha and beta chain variable domain pairings selected from the group consisting of: SEQ ID NO:1 with SEQ ID NOS:23-30 or 31; SEQ ID NOS:11-22 or SEQ ID NOS:50-60 with SEQ ID NO:2; SEQ ID NO:17 and SEQ ID NO:25; SEQ ID NO:17, 12, 16, or 14 with SEQ ID NO:23; SEQ ID NO:12 with SEQ ID NO:25; SEQ ID NO:15, 19, or 20 with SEQ ID NO:23; SEQ ID NO:11 with SEQ ID NO:61-64 or 65; SEQ ID NO:14, 17-19, 21, or 22 with SEQ ID NO:65; SEQ ID NO: 16 or 17 with SEQ ID NO:66; SEQ ID NO:11, 12, 16, 17, 19, or 20 with SEQ ID NO:31; SEQ ID NO:11 or 15 with SEQ ID NO:66; SEQ ID NO:11 or 17 with SEQ ID NO:67; SEQ ID NO:15 with SEQ ID NO:68; SEQ ID NO:15 with SEQ ID NO:8; SEQ ID NO:11, 15, or 17 with SEQ ID NO:69; SEQ ID NO:11, 15, or 17 with SEQ ID NO:70; and SEQ ID NO:11 with SEQ ID NO:23 or SEQ ID NO:28, optionally comprising one or more phenotypically silent substitutions.

62. A TCR as claimed in claim 37 wherein the polyalkylene glycol chain(s) comprise(s) at least two polyethylene glycol repeating units.

63. A method of treatment of cancer comprising administering to a subject suffering such cancer an effective amount of a TCR or a multivalent TCR complex as claimed in claim 1.

64. A method of treatment of cancer comprising administering to a subject suffering such cancer an effective amount of a plurality of cells as claimed in claim 55.


SEQUENCE LISTING (78 sequences)

SEQ ID NO: 1 - protein, 111 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVDSATSGTYKYIFGTGTRLKVLA

SEQ ID NO: 2 - protein, 111 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYQGTEAFFGQGTRLTVV

SEQ ID NO: 3 - DNA, 618 bp, Artificial Sequence
  DNA encoding soluble fragment of ILA TCR alpha chain
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggactctgctacctcaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 4 - DNA, 726 bp, Artificial Sequence
  DNA encoding soluble form of ILA TCR beta chain
aacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttaccaaggcactgaagctttctttggacaaggcaccagactcacagttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 5 - protein, 205 aa, Artificial Sequence
  Soluble fragment of ILA TCR alpha chain
GIQVEQSPPDLILQEGANSTLRCNFSDSVNNLQWFHQNPWGQLINLFYIPSGTKQNGRLSATTVATERYSLLYISSSQTTDSGVYFCAVDSATSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 6 - protein, 241 aa, Artificial Sequence
  Soluble fragment of ILA TCR beta chain
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYQGTEAFFGQGTRLTVVEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRAD

SEQ ID NO: 7 - DNA, 618 bp, Artificial Sequence
  DNA encoding soluble fragment of ILA TCR alpha chain with introduced cysteine
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggactctgctacctcaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 8 - DNA, 726 bp, Artificial Sequence
  DNA encoding soluble fragment of ILA TCR beta chain with introduced cysteine
aacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttaccaaggcactgaagctttctttggacaaggcaccagactcacagttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 9 - protein, 205 aa, Artificial Sequence
  Soluble fragment of ILA TCR alpha chain with introduced cysteine
GIQVEQSPPDLILQEGANSTLRCNFSDSVNNLQWFHQNPWGQLINLFYIPSGTKQNGRLSATTVATERYSLLYISSSQTTDSGVYFCAVDSATSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 10 - protein, 241 aa, Artificial Sequence
  Soluble fragment of ILA TCR with introduced cysteine
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYQGTEAFFGQGTRLTVVEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 11 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATALPYGYIFGTGTRLKVLA

SEQ ID NO: 12 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATQLPYGYIFGTGTRLKVLA

SEQ ID NO: 13 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATTLPYGYIFGTGTRLKVLA

SEQ ID NO: 14 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATGLPYGYIFGTGTRLKVLA

SEQ ID NO: 15 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATTGMFGYIFGTGTRLKVLA

SEQ ID NO: 16 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATSLPYGYIFGTGTRLKVLA

SEQ ID NO: 17 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATSPPYGYIFGTGTRLKVLA

SEQ ID NO: 18 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATLPPYGYIFGTGTRLKVLA

SEQ ID NO: 19 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATSIPYGYIFGTGTRLKVLA

SEQ ID NO: 20 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATSVPYGYIFGTGTRLKVLA

SEQ ID NO: 21 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATSGMFGYIFGTGTRLKVLA

SEQ ID NO: 22 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATTMPYGYIFGTGTRLKVLA

SEQ ID NO: 23 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPQGTEAFFGQGTRLTVV

SEQ ID NO: 24 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGLQPQGTEAFFGQGTRLTVV

SEQ ID NO: 25 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGVQPQGTEAFFGQGTRLTVV

SEQ ID NO: 26 - protein, 111 aa, Artificial Sequence
  High affinity variant of ILA TCR beta chain variable domain
NAGVTQTPKFQVLKTGQSVTLQCAQDMNHEYMSWYRQDPGMGLRLIHYSIHPEYTDQGEVPNGYNVSRSTTEDFPLRLLSAAPSQTSVYFCASSYQGTEAFFGQGTRLTVV

SEQ ID NO: 27 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSLHPSVTDQGEVPNGYNVSRSTTEDFPLRLLSAAPSQTSVYFCASSYQGTEAFFGQGTRLTVV

SEQ ID NO: 28 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSICPSCTDQGEVPNGYNVSRSTTEDFPLRLLSAAPSQTSVYFCASSYQGTEAFFGQGTRLTVV

SEQ ID NO: 29 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSICWGCTDQGEVPNGYNVSRSTTEDFPLRLLSAAPSQTSVYFCASSYQGTEAFFGQGTRLTVV

SEQ ID NO: 30 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSIWEFETDQGEVPNGYNVSRSTTEDFPLRLLSAAPSQTSVYFCASSYQGTEAFFGQGTRLTVV

SEQ ID NO: 31 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSRWVGDTDQGEVPNGYNVSRSTTEDFPLRLLSAAPSQTSVYFCASSYQGTEAFFGQGTRLTVV

SEQ ID NO: 32 - protein, 47 aa, Homo sapiens
NIQNPDPAVYQLRDSKSSDKSVCLFTDFDSQTNVSQSKDSDVYITDK

SEQ ID NO: 33 - protein, 56 aa, Homo sapiens
EDLNKVFPPEVAVFEPSEAEISHTQKATLVCLATGFFPDHVELSWWVNGKEVHSGV

SEQ ID NO: 34 - protein, 56 aa, Homo sapiens
EDLKNVFPPEVAVFEPSEAEISHTQKATLVCLATGFYPDHVELSWWVNGKEVHSGV

SEQ ID NO: 35 - protein, 30 aa, Artificial Sequence
  Short scTCR interchain linker
PGGGSGGGGSGGGGSGGGGSGGGGSGGGGP

SEQ ID NO: 36 - protein, 35 aa, Artificial Sequence
  Long TCR interchain linker
PGGGSGGGGSGGGGSGGGGSGGGGSGGGGSGGGGP

SEQ ID NO: 37 - protein, 9 aa, Homo sapiens
ILAKFLHWL

SEQ ID NO: 38 - DNA, 37 bp, Artificial Sequence
  primer
tctctcattaatgggaatacaagtggagcagagtcct

SEQ ID NO: 39 - DNA, 20 bp, Artificial Sequence
  Primer
cggcagggtcagggttctgg

SEQ ID NO: 40 - DNA, 20 bp, Artificial sequence
  Primer
ccagaaccctgaccctgccg

SEQ ID NO: 41 - DNA, 30 bp, Artificial Sequence
  Primer
aagcttcccgggggaactttctgggctggg

SEQ ID NO: 42 - DNA, 36 bp, Artificial Sequence
  Primer
tctctcattaatgaatgctggtgtcactcagacccc

SEQ ID NO: 43 - DNA, 22 bp, Artificial Sequence
  Primer
cttctgatggctcaaacacagc

SEQ ID NO: 44 - DNA, 22 bp, Artificial Sequence
  Primer
gctgtgtttgagccatcagaag

SEQ ID NO: 45 - DNA, 30 bp, Artificial Sequence
  Primer
aagcttcccggggtctgctctaccccaggc

SEQ ID NO: 46 - DNA, 3342 bp, Artificial Sequence
  pEX954 plasmid
gatctcgatcccgcgaaattaatacgactcactatagggagaccacaacggtttccctctagaaataattttgtttaactttaagaaggagatataatcgatgtctaactcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtgaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 47 - DNA, 3836 bp, Artificial Sequence
  pEX821 plasmid
gatctcgatcccgcgaaattaatacgactcactatagggagaccacaacggtttccctctagaaataattttgtttaactttaagaaggagatatacatatgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 48 - DNA, 3857 bp, Artificial Sequence
  pEX202 plasmid
gatctcgatcccgcgaaattaatacgactcactatagggagaccacaacggtttccctctagaaataattttgtttaactttaagaaggagatatacatatgcagaaggaagtggagcagaactctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgttacaactgacagctgggggaaattgcagtttggagcagggacccaggttgtggtcaccccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcccccgggggtagaatcgcccggctggaggaaaaagtgaaaaccttgaaagctcagaactcggagctggcgtccacggccaacatgctcagggaacaggtggcacagcttaaacagaaagtcatgaactactaggatccatggtaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 49 - DNA, 4019 bp, Artificial Sequence
  pEX205 plasmid
gatctcgatcccgcgaaattaatacgactcactatagggagaccacaacggtttccctctagaaataattttgtttaactttaagaaggagatatacatatgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagaccccgggggtctgactgatacactccaagcggagacagatcaacttgaagacaagaagtctgcgttgcagaccgagattgccaatctactgaaagagaaggaaaaactagagttcatcctggcagcttacggatccggtggtggtctgaacgatatttttgaagctcagaaaatcgaatggcattaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 50 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATMPTGKYIFGTGTRLKVLA

SEQ ID NO: 51 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATRLPFLYIFGTGTRLKVLA

SEQ ID NO: 52 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATQLPFLYIFGTGTRLKVLA

SEQ ID NO: 53 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATMLPYGYIFGTGTRLKVLA

SEQ ID NO: 54 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATALPFGYIFGTGTRLKVLA

SEQ ID NO: 55 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATALPYAYIFGTGTRLKVLA

SEQ ID NO: 56 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATHLPYGYIFGTGTRLKVLA

SEQ ID NO: 57 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATTPPYGYIFGTGTRLKVLA

SEQ ID NO: 58 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATEMPYGYIFGTGTRLKVLA

SEQ ID NO: 59 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATNLPYGYIFGTGTRLKVLA

SEQ ID NO: 60 - protein, 111 aa, Artificial Sequence
  High affinity variant of ILA TCR alpha chain variable domain
GIQVEQSPPDLILQEGANSTLRCNFSDSVNNLQWFHQNPWGQLINLFYIPSGTKQNGRLSATTVATERYSLLYISSSQTTDSGVYFCAVDSATKLPYGYIFGTGTRLKVLA

SEQ ID NO: 61 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSIHPEYTDQGEVPNGYNVSRSTTEDFPLRLLSAAPSQTSVYFCGIQPQGTEAFFGQGTRLTVV

SEQ ID NO: 62 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSICPSCTDQGEVPNGYNVSRSTTEDFPLRLLSAAPSQTSVYFCGIQPQGTEAFFGQGTRLTVV

SEQ ID NO: 63 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSIHPEYTDQGEVPNGYNVSRSTTEDFPLRLLSAAPSQTSVYFCGVQPQGTEAFFGQGTRLTVV

SEQ ID NO: 64 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSICPSCTDQGEVPNGYNVSRSTTEDFPLRLLSAAPSQTSVYFCGVQPQGTEAFFGQGTRLTVV

SEQ ID NO: 65 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSRWVGDTDQGEVPNGYNVSRSTTEDFPLRLLSAAPSQTSVYFCGVQPQGTEAFFGQGTRLTVV

SEQ ID NO: 66 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSRWVGDTDQGEVPNGYNVSRSTTEDFPLRLLSAAPSQTSVYFCGIQPQGTEAFFGQGTRLTVV

SEQ ID NO: 67 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSICWGCTDQGEVPNGYNVSRSTTEDFPLRLLSAAPSQTSVYFCGIQPQGTEAFFGQGTRLTVV

SEQ ID NO: 68 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSIWEFETDQGEVPNGYNVSRSTTEDFPLRLLSAAPSQTSVYFCGIQPQGTEAFFGQGTRLTVV

SEQ ID NO: 69 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSIWEFETDQGEVPNGYNVSRSTTEDFPLRLLSAAPSQTSVYFCGVQPQGTEAFFGQGTRLTVV

SEQ ID NO: 70 - protein, 111 aa, Artificial Sequence
  High affinity variant of ILA TCR beta chain variable domain
NAGVTQTPKFQVLKTGQSMTLQCAQDMNHEYMSWYRQDPGMGLRLIHYSICWGCTDQGEVPNGYNVSRSTTEDFPLRLLSAAPSQTSVYFCGVQPQGTEAFFGQGTRLTVV

SEQ ID NO: 71 - protein, 205 aa, Artificial Sequence
  Soluble DiS-linked high affinity variant of ILA TCR alpha chain
MGIQVEQSPPDLILQEGANSTLRCNFSDSVNNLQWFHQNPWGQLINLFYIPSGTKQNGRLSATTVATERYSLLYISSSQTTDSGVYFCAVDSATALPYGYIFGTGTRLKVLAIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 72 - protein, 378 aa, Artificial Sequence
  Soluble DiS-linked high affinity variant of ILA TCR beta chain linked to wild-type human IL-2
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPYQGTEAFFGQGTRLTVVEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADPGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 73 - protein, 243 aa, Artificial Sequence
  Soluble DiS-linked high affinity variant of ILA TCR beta chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPYQGTEAFFGQGTRLTVVEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 74 - protein, 243 aa, Artificial Sequence
  Soluble DiS-linked high affinity variant of ILA TCR beta chain
MNAGVTQTPKFQVLKTGQSMTLQCAQDMNHEYMSWYRQDPGMGLRLIHYSICPSCTDQGEVPNGYNVSRSTTEDFPLRLLSAAPSQTSVYFCGVQPYQGTEAFFGQGTRLTVVEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 75 - protein, 243 aa, Artificial Sequence
  Soluble DiS-linked high affinity variant of ILA TCR beta chain
MNAGVTQTPKFQVLKTGQSMTLQCAQDMNHEYMSWYRQDPGMGLRLIHYSRWVGDTDQGEVPNGYNVSRSTTEDFPLRLLSAAPSQTSVYFCGVQPYQGTEAFFGQGTRLTVVEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 76 - protein, 243 aa, Artificial Sequence
  Soluble DiS-linked high affinity variant of ILA TCR beta chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPYQGTEAFFGQGTRLTVVEDLNKVFPPEVAVFEPSEAEISHTQKATLVCLATGFFPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 77 - protein, 243 aa, Artificial Sequence
  Soluble DiS-linked high affinity variant of ILA TCR beta chain
MNAGVTQTPKFQVLKTGQSMTLQCAQDMNHEYMSWYRQDPGMGLRLIHYSICPSCTDQGEVPNGYNVSRSTTEDFPLRLLSAAPSQTSVYFCGVQPYQGTEAFFGQGTRLTVVEDLNKVFPPEVAVFEPSEAEISHTQKATLVCLATGFFPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 78 - protein, 243 aa, Artificial Sequence
  Soluble DiS-linked high affinity variant of ILA TCR beta chain
MNAGVTQTPKFQVLKTGQSMTLQCAQDMNHEYMSWYRQDPGMGLRLIHYSRWVGDTDQGEVPNGYNVSRSTTEDFPLRLLSAAPSQTSVYFCGVQPYQGTEAFFGQGTRLTVVEDLNKVFPPEVAVFEPSEAEISHTQKATLVCLATGFFPDHVELSWWVNGKEVHSGVCTDPQPLKEQPALNDSRYALSSRLRVSATFWQDPRNHFRCQVQFYGLSENDEWTQDRAKPVTQIVSAEAWGRAD